(19) United States
(12) Patent Application Publication
(10) Pub. No.: US 2021/0052221 A1
(43) Pub. Date: Feb. 25, 2021

(54) SYSTEM, METHOD, AND SMARTWATCH FOR PROTECTING A USER

(71) Applicant: VitalTech Properties, LLC, Plano, TX (US)

(72) Inventors: Anjan Panneer Selvam, Plano, TX (US); Peter Ianace, Frisco, TX (US)

(73) Assignee: VitalTech Properties, LLC, Plano, TX (US)

(21) Appl. No.: 17/000,035

(22) Filed: Aug. 21, 2020

Related U.S. Application Data
(60) Provisional application No. 62/890,847, filed on Aug. 23, 2019.

(51) Int. Cl. A61B 5/00; A61B 5/11

(52) U.S. Cl. CPC A61B 5/681; A61B 5/1112; A61B 5/1118; A61B 5/4866; A61B 5/746; A61B 5/7282; A61B 5/4875; A61B 5/486

(57) ABSTRACT

A system, method, and smartwatch for protecting a user. Daily living metrics and behavioral metrics are measured utilizing a smartwatch worn by a user. Metrics including the daily living metrics and the behavioral metrics are compiled. One or more scores are generated for daily living and behavioral utilizing the metrics. One or more alerts are communicated for one or more authorized users in response to the one or more scores.

### PRIORITY

This application claims priority to U.S. Provisional Patent Application Ser. No. 62/890,847 filed on Aug. 23, 2019, which is incorporated by reference herein in its entirety.

### BACKGROUND

### I. Field of the Disclosure

The illustrative embodiments relate to biometrics and risk analysis. More specifically, but not exclusively, the illustrative embodiments relate to a system, method, smartwatch, server, computer program product, and wearable for monitoring a user's well-being.

### II. Description of the Art

Each year thousands of people suffer injuries and trauma due to a falling event. The risk of suffering falling events is even higher for elderly, incapacitated, or sick individuals. There are increasing number of tools to detect a falling event. Despite the increases in technology, medical providers, caregivers, organizations, facilities, and others still struggle to prevent falling events.

In addition, there is a large portion of the world population that knowingly or unknowingly enters a state of dehydration on a daily, weekly, or monthly basis. The hydration may occur due to physical exertion, such as extensive sports activities. Dehydration is also common in sedentary environments, such as office workplaces, college classrooms, and other similar locations or situations. This type of dehydration is often imperceptible to the user experiencing it. As is well known, dehydration has a wide range of adverse effects on human physiology (e.g., physical performance decreases, impaired cognitive functions, irritability, organ operation, etc.). Despite improvements in technology, medical providers, caregivers, organizations, facilities, and others still struggle to monitor users.

### SUMMARY

The illustrative embodiments provide a system, method, and smartwatch for protecting a user. Daily living metrics and behavioral metrics are measured utilizing a smartwatch worn by a user. Metrics including the daily living metrics and the behavioral metrics are compiled. One or more scores are generated for daily living and behavioral utilizing the metrics. One or more alerts are communicated for one or more authorized users in response to the one or more scores.

In another embodiment, the illustrative embodiments provide a system, method, and smartwatch for determining an impact threshold score. Inputs for height, weight, activity level, hydration information, and age of a user are received. A body mass index of the user is determined. Values for the activity level, body mass index, hydration information, and the age of the user are assigned. The impact threshold score is calculated utilizing the values. Thresholds for a smartwatch are established in response to the impact threshold score.

In other embodiments, the inputs may be received audibly utilizing at least the smartwatch. The inputs may also be received from any number of external devices, systems, equipment, or components. The inputs may be received through the user interface of the smartwatch including at least one or more buttons, a microphone, and a touch screen. The user may be prompted to provide the inputs for height, weight, and age of the user and the activity level and hydration information for the user may be detected utilizing one or more sensors of the smartwatch. One or more optical and conductivity tests may be performed on the user utilizing the smartwatch, the measurements from the test may be compiled, and the measurements may be analyzed to generate the hydration information regarding the user. A menu of options may be presented to the user or a person associated with the user to receive the inputs. The values received from the user may be normalized when assigning the values. A fall prediction assessment may be generated utilizing the thresholds and impact threshold score. Biometrics from the user may be measured utilizing at least the sensors of the smartwatch and an alert may be communicated to the user or one or more authorized parties in response to the biometrics of the user exceeding the thresholds. A hydration mode of the smartwatch may be activated, a determination of whether the user is hydrated may be made, characteristics of the skin of the user may be measured in response to determining the user is hydrated, and a hydration profile for the user may be created utilizing the characteristics for subsequently determining the hydration information. An interior surface and an exterior surface of the smartwatch may include electrodes for determining the hydration information of the user.

Another illustrative embodiment provides a smartwatch for monitoring a user. The smartwatch includes a band securing the smartwatch to the arm of the user. The smartwatch further includes a body of the smartwatch housing logic and at least a portion of one or more sensors the one or more sensors measure biometrics, an activity level, and hydration information of the user. The smartwatch further includes a user interface in communication with the logic configured to receive physiological parameters from the user. The logic determines a body mass index of the user utilizing the physiological parameters, assign values to the activity level, hydration information, and the physiological parameters, calculates an impact threshold score utilizing the values, and establishes thresholds for the smartwatch in response to the impact threshold score.

In other embodiments, the physiological parameters may include at least height, weight, and age of the user. The physiological parameters may be received as input through a user interface of the smartwatch. The interface may include at least a microphone, one or more touch sensors, and one or more buttons. The logic generates one or more alerts for communication through the user interface in response to the biometrics exceeding one or more of the thresholds. The alerts may be sent to one or more authorized devices or users associated with the user. The one or more sensors include one or more optical sensors for measuring the biometrics and conductivity sensors for measuring the hydration information.

Another illustrative embodiment provides a system for monitoring a user. The system includes a server configured to communicate through one or more networks. The server receives inputs for at least height, weight, and age of a user. The system further includes a smartwatch that measures an activity level and hydration for the user utilizing one or more sensors of the smartwatch and communicates the activity level and hydration information from a transceiver of the smartwatch through one or more networks to the server. The server determines a body mass index of the user utilizing the height and weight of the user, assign values for activity level, body mass index, hydration information, and the age of the user, calculates an impact threshold score and thresholds utilizing the values, and communicates the thresholds to the smartwatch for monitoring the biometrics of the user to prevent falls.

In other embodiments, the thresholds may prevent health or medical events associated with the user. The smartwatch may utilize the thresholds to monitor the user and the smartwatch generates one or more alerts for the user and/or authorized users in communication with the user through the transceiver and one or more networks. The smartwatch may utilize optical measurements and/or conductivity measurements from the one or more sensors to determine the activity level, hydration information, and biometrics of the user. The activity level may be determined at least based on motion of the user throughout a time period. The biometrics may be compared against baseline readings for the user. The smartwatch may receive a SIM card for communicating through one or more cellular networks. The smartwatch includes a rechargeable battery.

### BRIEF DESCRIPTION OF THE DRAWINGS

Illustrated embodiments are described in detail below with reference to the attached drawing figures, which are incorporated by reference herein, and where:

FIG. 1 illustrates a perspective view of a smartwatch in accordance with an illustrative embodiment;

FIG. 2 is a front view of the smartwatch of FIG. 1 in accordance with an illustrative embodiment;

FIG. 3 is a rear view of the smartwatch of FIG. 1 in accordance with an illustrative embodiment;

FIGS. 4-5 are side views of the smartwatch of FIG. 1 in accordance with an illustrative embodiment;

FIG. 6 is a front view and side vies of another smartwatch in accordance with an illustrative embodiment;

FIGS. 7-10 are a partial view of the smartwatch of FIG. 6 in accordance with an illustrative embodiment.

FIG. 11 is a pictorial representation of users wearing a smartwatch in accordance with an illustrative embodiment;

FIG. 12 is a pictorial representation of a block diagram of a smartwatch in accordance with an illustrative embodiment;

FIG. 13 is a pictorial representation of modules utilized by the smartwatch of FIG. 12 in accordance with an illustrative embodiment;

FIG. 14 is another block diagram of a smartwatch 1400 in accordance with an illustrative embodiment;

FIG. 15 is a flowchart of a process for generating an automated score decision based on thresholds in accordance with an illustrative embodiment;

FIG. 16 is a flowchart of a process for creating a hydration profile in accordance with an illustrative embodiment;

FIG. 17 is a flowchart of a process for performing hydration monitoring in accordance with an illustrative embodiment;

FIG. 18 is a flowchart of a process for automatically performing hydration monitoring in accordance with an illustrative embodiment;

FIG. 19 is a pictorial representation of a smartwatches with enhanced bands in accordance with an illustrative embodiment;

FIG. 20 is a pictorial representation of a smartwatch measuring hydration in accordance with an illustrative embodiment; and

FIG. 21 depicts a computing system in accordance with an illustrative embodiment;

FIG. 22 is a pictorial representation of a flowchart for determining a baseline score for a user in accordance with an illustrative embodiment;

FIG. 23 is a pictorial representation of a flowchart for generating one or more scores for daily living and behavior in accordance with an illustrative embodiment;

FIG. 24 is a pictorial representation of a flowchart communicating recommendations and alerts in accordance with an illustrative embodiment; and

FIG. 25 is a pictorial representation of a flowchart for providing recommendations in accordance with an illustrative embodiment.

### DETAILED DESCRIPTION OF THE DISCLOSURE

The illustrative embodiments provide a system, method, device, smartwatch, smart band, server, computer readable instructions, or biosensing wearable for monitoring a user. The user is described herein may represent elderly, young, infirm, disabled, or at-risk individuals that are being monitored for their own protection, safety, and well-being. However, the smartwatch may also be utilized for any user to better provide for their needs and well-being.

Any number of daily living metrics and behavioral metrics may be monitored to generate a score. The score may be monitored to protect the well-being of the user (as well as those around the user). Baseline measurements may be taken to determine an initial status and condition of the user. The baseline measurements may also provide any default standard baseline against which additional metrics may be measured. For example, once a baseline score is established. Additional metrics read by the smartwatch, sensors, devices, components, and/or associated system.

The user may be periodically given a daily living score and/or behavioral score that assesses the care, self-care, and general well-being of the user. The scores may be utilized to provide recommendations, changes, and alerts to the user or those associated with the user (e.g., caregivers, family, medical professionals, authorized persons, etc.). User information, such as motion, location, and activities may be utilized to customize the feedback, recommendations, and/or alerts that are provided to the user/authorized users.

The illustrative embodiments may also be utilized to address conditions or events. For example, the smartwatch may be utilized to provide recommendations for handwashing, social distancing, changes in activities, or other user actions that could be beneficial to the user to prevent contracting or spreading diseases, such as the 2020 spread of COVID-19. The various embodiments may also be utilized to protect the user from environmental conditions, such as air contamination, bacterial infections, allergies, and so forth.

In addition, any number of impact threshold scores and fall risk assessments may also be utilized to perform fall likelihood monitoring. The user may represent any number of patients, elderly individuals, persons with disabilities, special needs individuals, regular users, children, or others. As used herein, smartwatch may refer to any device that is worn, adhered to, or positioned on or within the body of the user including bands, sensor modules, anklets, straps, stickers, or other devices. For example, the smartwatch may represent any number of smart wearables that are integrated with or connected to an attachment mechanism, such as a band, strap, sticker, clothing, or so forth. The smartwatch may be worn on the user's wrist, arm, head, neck, leg, chest, shoulder, or so forth.

The smartwatch may be especially beneficial for monitoring at-risk individuals, such as the elderly, sick, or children. These individuals may be particularly susceptible to falls, becoming lost, being abducted/kidnapped, suffering from dangerous medical conditions, or other potentially problematic issues. The smartwatch may be utilized to monitor the user, provide notifications/reminders/alerts, monitor fitness information and biometrics, and send and receive emergency messages.

The smartwatch may be utilized to perform biometric, activity, and location-based monitoring. The smartwatch may provide alerts, notifications, or other indicators to the wearer of the smartwatch as well as any number of other users or devices. For example, emergency alerts may be generated automatically or in response to feedback from the user for protecting the user.

The sensors of the smartwatch including accelerometers, gyroscopes, magnetometers, optical sensors, touch sensors, time-of-flight sensors, blood pressure, blood oxygenation, hydration, mechanical sensors, piezo electric sensors, heart rate, retinal sensors, fingerprint sensors, pressure sensors, microphones, temperature sensors, and water sensors (for one or more of the user, environment, and smartwatch itself). For example, parameters extracted by an optical sensor (e.g., emitter, receiver, etc.) may include, but are not limited to, heart rate, arrhythmias, heart rate variability (HRV), preventricular contractions (PVC), tachycardia, bradycardia, dehydration, and so forth.

The feedback and output devices of the smartwatch may be utilized to provide different stimuli and feedback to the user. In one embodiment, a screen, current/voltage generator, speakers, or a vibration component may be utilized to provide feedback and instructions to the user. As a result, the user may receive information regardless of their capacity to speak or provide verbal or tactile feedback.

The illustrative embodiments are unique in that the smartwatch or wearable may be worn by the user. As a result, the user does not have to hold a device, remember to perform biometric readings, or remember to include an alert device. The various sensors of the smartwatch may work alone and in combination to accurately determine user and environmental information. The smartwatch may score the user's biometrics and status. The smartwatch may be used as a stand-alone device or with other electronic devices (e.g., cell phones, personal computers, etc.) wearables (e.g., hearables, exercise equipment, etc.) or implantable devices. As previously noted, the smartwatch may be worn directly on the user or may be integrated with a shirt, hat, shoe, bag, or other article of clothing or accessory worn or carried by the user.

The smartwatch may generate alerts in response to biometric readings of the user exceeding one or more thresholds. Alerts may also be generated based on the location of the user. For example, a geo-fence may be established around expected locations for the user with alerts generated if the smartwatch is detected outside of those geofenced areas. The user may send an SOS message in response to covering the smartwatch, squeezing one or more portions of the face of the smartwatch or button sequences, tapping the smartwatch in a pattern, or providing other specified input. The command to send an SOS message is as simple as possible to allow an injured, disabled, or otherwise incapacitated user to send the SOS communication.

In one embodiment, the smartwatch may include a locking band. The locking band may be utilized to ensure that the user cannot remove the smartwatch without permission or authorization. For example, the locking band may include a locking mechanism that requires a specific tool to be locked and unlocked. The locking band may also use a digital authorization or signal two lock and unlock the locking band. The locking band may include anti-removal hardware. For example, the band may include wires or conductors that if cut or broken automatically generate an alert. The band may also include a regular latching or securing mechanism that sends an alert when removed from the user. The alert may include sending a message with the last known location and status of the user, generating an audio alert, and sending any number of messages to authorized devices/users.

The smartwatch is utilized to detect and predict falls based on a user's present, past, and expected physiological factors and parameters. The illustrative embodiments provide a process for determining an impact threshold score. The score may be determined utilizing height, weight, activity level, and age of a patient. The body mass index (BMI) of the patient may be calculated utilizing available information or utilizing the smartwatch. Values may be assigned for the activity level, BMI, and the age of the patient. The impact threshold score may be calculated utilizing the values. The smartwatch establishes thresholds for the biosensing wearable in response to the impact threshold score. The thresholds may be utilized to predict or determine when a fall is imminent, likely, or risks are elevated.

The illustrative embodiments provide processes for determining fall risks predictions, assessment, and detection of a user. Additional fall risks factors may include physical condition, time of day, medication consumption, heart range changes, and other applicable information internal or external to the user. The input devices of the smartwatch may receive user input and measure applicable biometric and environmental data and information. The input information, data, and parameters utilized by the smartwatch may include age, height, weight, BMI, activity level, waist size, length of limbs, skin pore density, heart rate, respiration rate, blood pressure, arterial pressure, blood oxygenation, skin resistance, bone density, orientation with respect to center of gravity (e.g., resting and active), dominant hand, dominant foot, feet posture, and other medically relevant data. The output devices of the smartwatch may be utilized to provide different stimuli and feedback to the user. In one embodiment, a vibrator may be utilized to provide instructions to the user. The display, light emitting diodes, speakers, electrical contacts, or other components may also be utilized to provide feedback to the user.

The user does not have to focus on holding a device or ensuring a proper biometric interface. The biosensing wearable of the illustrative embodiments may represent a smart watch, bracelet, helmet, hearing aid, sticker, patch, band, or other smart wearable. The biosensing wearable may be worn on a wrist, ankle, arm, leg, hip, neck, or any number of joints. In another embodiment, the biosensing wearable may be a miniature device encompassed in a case protecting the electrical components. The biosensing wearable be slipped or integrated into a pocket, sleeve, or other aspect of a user's clothing or accessories. In addition, the sensors of the biosensing wearable may work together to provide an impact threshold score, assessments, thresholds, predictions, and alerts. The impact threshold score, data, or information may be utilized to provide special care, monitoring, or resources for a patient/user that may be in danger of experiencing a fall event.

As noted, the smartwatch may be utilized alone, in multiples (e.g., left wrist, right wrist, etc.), on different appendages (e.g., ankle mounted, chest mounted, etc.), in different configurations, or so forth. In one embodiment, the biosensing wearable may be integrated with a shirt, hat, shoe, bag, or other article of clothing or accessory worn or carried by the user. The biosensing wearable may also represent a smartwatch, smart sensor array, smart bracelet, smart sticker, smart headband, smart clothing, smart implantable or so forth. The biosensing wearable may be rechargeable and reusable or disposable/single-use.

In some embodiments, individuals assessed (i.e., users or subjects) may be divided into four groups, including, but not limited to, community dwelling, extended term care facility, hospitalized, and the cognitively impaired. Other individuals with diseases, impairments, disabilities, or conditions may also be evaluated. In some embodiments, fall risk may be better assessed by the user's spouse, partner, responsible family member, caregiver, or healthcare professional. In one embodiment, the illustrative embodiments may represent an advanced individualized fall risk assessment test (AIFRAT). The testing and assessment as described may be performed in 15-30 minutes or less depending on the stamina and cooperation of the user with rest periods added as needed for the patient and data recordation. The individuals may be stratified into higher level fall risk groups (e.g., Level I, II, III) and multifunctional interventions may be suggested. The scoring system utilized for assigning levels may reflect both observational and objective risk stratification. Suggestions for intervention may include strength training, balance and stability exercises, modified home environments, and/or reduced polypharmacy or psychotropic medications. The various types of tests, examinations, and analysis may cover cognition, strength, balance and stability, gait, and functional performance.

In one embodiment, the examination may allow for the integration of questions and physical tests. The test may be implemented progressively with a basic level of success in each category or test being required before proceeding on to the next. Referrals for more detailed assessments may be given for those who do not successfully pass a level, despite one or more attempts. The illustrative embodiments allow the objective analysis based on data and measurements from one or more wearable devices for information, such as vital signs, balance and sway, gait, and other applicable movements. The results may suggest guide interventions, establish baselines, and allow for comparisons over time.

For example, the testing and analysis may be referred to as wearable sensor-based Advanced Fall Risk Assessment Testing (AFRAT). In one embodiment, the illustrative embodiments provide a method of identifying individuals at “high risk” for a potential fall. The various tests may be performed for individuals 65 or older or those identified as having high risk of falling. The risks may result from frailty, fear of falling, neurologic disease, musculoskeletal disease, use of a mobility assistance device, disorientation, vertigo, and so forth. The biosensing wearable includes an array of sensors for detecting information about the user as well as the user's environment. The sensors of the biosensing wearable including accelerometers, gyroscopes, and optical sensors. For example, parameters extracted by the optical sensor (e.g., emitter, receiver, etc.) may include, but are not limited to, heart rate variability (HRV), heart rate, arrhythmias, preventricular contractions (PVC), tachycardia, bradycardia, and so forth.

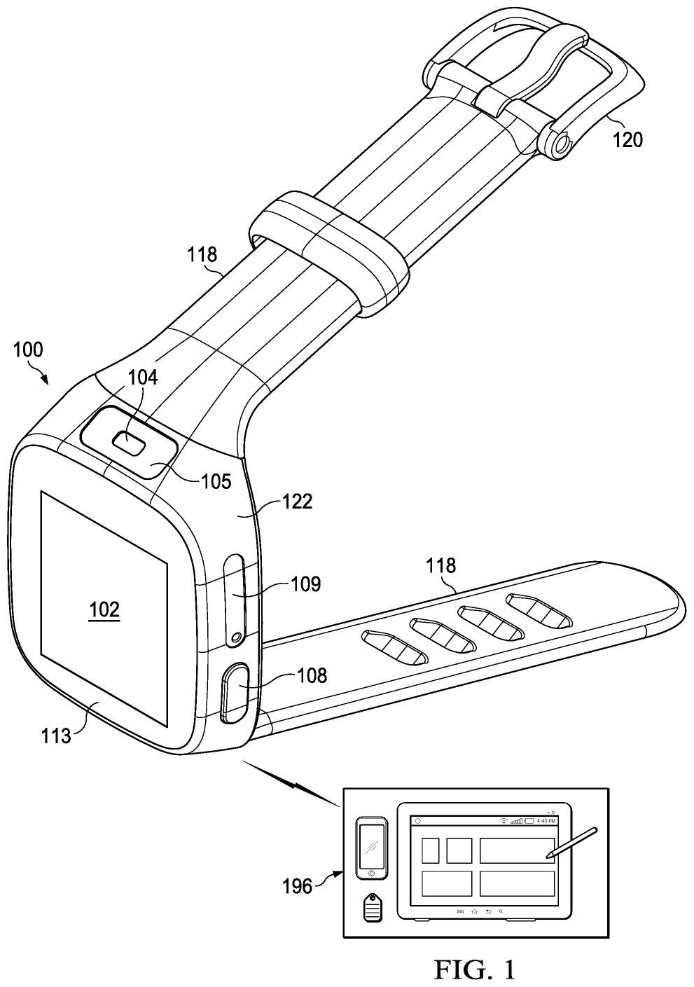

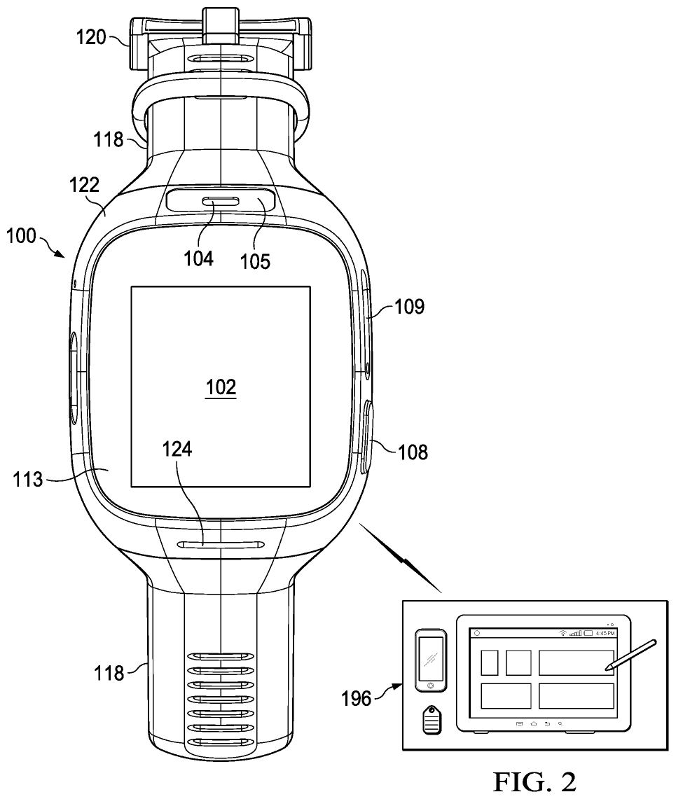

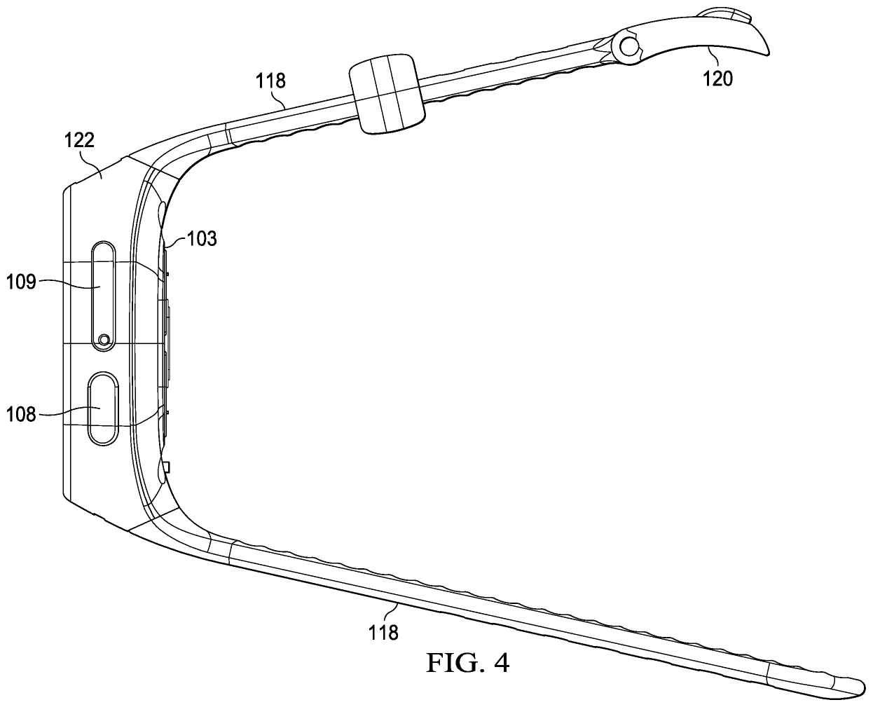

Turning now to FIGS. 1-5 illustrating distinct views of a smartwatch 100 in accordance with an illustrative embodiment. The smartwatch may include a display 102, a optical sensor 104, electrode 105, electrodes 106, sensor array 107, button 108, SIM card slot 109, speaker 110, microphone 112, charging pins 114, heart rate sensor 116, a band 118, a latch 120, a housing 122, and an external sensor 124.

The smartwatch 100 has a housing 122 configured to be positioned against the user's body and the band 118 configured to hold the housing 122 against the user's body or skin. The housing 122 is shaped and sized to fit on the desired target location for wearing the smartwatch 100, such as on the wrist, ankle, neck, head, leg, or upper-arm of the user. The housing 122 is a protective case, shell, or platform to which the remaining parts are directly or indirectly attached, integrated, or housed. A plastic or metallic housing structure is expected to be suitable for most embodiments. For example, the housing 122 may be injection-molded plastic, cast magnesium, machined aluminum or steel, a polymer, or other strong materials. The housing 122 may be molded, 3D printed, machined, or otherwise generated. The housing 122 may include seals and other waterproof components. The housing 122 provides a framework that encases and protects the components of the smartwatch 100. In one embodiment, the smartwatch 100 is waterproof or water resistant so that the user can wear the smartwatch 100 is any number of locations, circumstances or during activities where it is important to monitor the user (e.g., bathroom, bath, exercising, etc.).

In one embodiment, all, or portions of the housing 122 may be removed to access, fix, replace, or update various portions of the smartwatch 100. For example, a back of the housing 122 may include a removeable plate that may be attached utilizing miniatures bolts/screws that are sealed utilizing one or more seals. As a result, components, such as the battery, transceiver, buttons, logic, memory, display, or so forth may be replaced as needed.

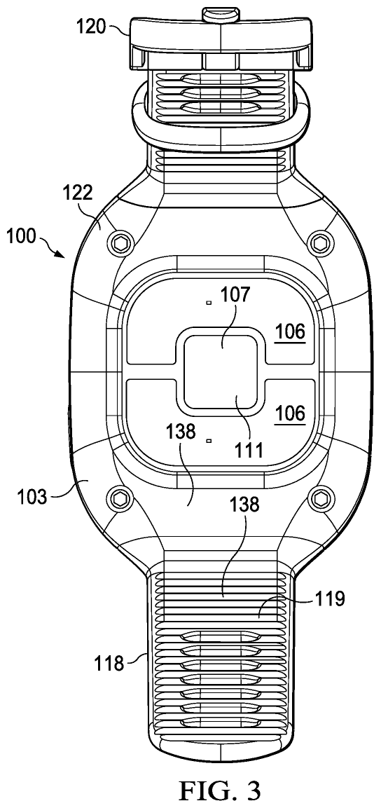

The band 118 may attach to or extend from edges of the housing utilizing one or more pins, rods, supports, or so forth. The band(s) 118 may include a support or structure that wraps entirely or partially around the wearer's body to hold smartwatch 100 in place. In the example shown, the band 118 is configured to hold the housing 122 against the wearer's wrist, ankle, or shoulder. The band 118 may also be configured to hold the housing against the head, upper arm, hand, finger, leg, neck, waist, chest, ankle, leg, or additional portion of the user's body. The band 118 may include one or more flexible or rigid straps. The band 118 may also utilize a spring-loaded or interference fit. One or more antennas of the smartwatch may extend partially or completely into the band 118. For example, one or more antennas may extend into a portion 119 of the band (see FIG. 3). For example, the portion 119 may extend from one or both sides of the band 118 for one or more of a cellular, Wi-Fi, Bluetooth, or other proprietary signals, protocols or standards utilized by the smartwatch 100.

In one embodiment, the band 118 may be secured, closed, bound, or joined joinable by a latch 120. The latch 120 may represent a buckle, magnets, buttons, or other securing and locking mechanism. The latch 120 may be self-closed or may be secured to the body or clothing of the user. In one embodiment, the latch 120 may lock the band 118 to the body so that it may be only removed utilizing a secured process (e.g., key, digital code, magnetic release, radio frequency tag, etc.). For example, the latch 120 may be utilized to secure the smartwatch 100 to a user with memory issues, a medical condition, a tendency to wander, an infant or child, or others that may need special care. As a result, the user may be more carefully monitored.

In one embodiment, the band 118 may include conductors or other sensors. If cut, broken, or removed without the latch 120 being properly opened, the smartwatch 100 may generate an alert. The smartwatch 100 may also send a running record of the status and location of the user in case the smartwatch 100 is removed, damaged, or purposely destroyed so that the data is kept in a database, cloud system, server, or other device. For example, the status of the user may be sent at a pre-defined interval, such as 30 seconds, 1 minute, 5 minutes, 10 minutes, 30 minutes, 1 hour, or other time period.

The band 118 may be movably or rigidly secured to the housing 122 by pivot pins, a cantilevered anchor, and so on. The band 118 also may be composed or formed integrally with the housing 122. The band 118 may also be removed for securing the body/housing 122 of the smartwatch 100 (without the band 118) within or to clothing, adhesives, third party straps, or so forth. For example, the housing 122 may be directly adhered to the body of the user.

In one embodiment, when configured for use on the wrist, the smartwatch 100 may have the form factor of a traditional watch or fitness tracker. For example, the smartwatch 100 may be formed as a watch, band, or strap. The components of the smartwatch 100 may be integrated into all or portions of the housing 122 and/or band 118. For example, the smartwatch 100 may not include a traditional larger display, but instead may have a smaller display or displays that fits into the narrower band 118. An interior portion of the housing 122 may include any number of dividers, separators, walls, slots, mounts, or other components for manufacturing, replacing, or separating the components of the smartwatch 100.

In one embodiment, the band 118 may house flexible electronics. The flexible electronics and circuits may be mounted on a flexible plastic substrate, such as polyimide, PEEK, or transparent conductive polyester film. Various photolithographic techniques may be used to generate the various components and electronics as herein described. In one example, the housing 122 may have a generally flat rectangular or rounded shape (e.g., rectangle, circle, ellipse, etc.) that extends in a plane with a maximum dimension in the plane of approximately two inches or less, and a thickness extending perpendicular to the plane of approximately one quarter inch or less. However, the dimensions of the smartwatch 100 may vary. The band 118 may be rotationally attached to edges of the housing 122 and configured to encircle a volume having a diameter of about two to three inches (e.g., circumference of 6.5-9.5 inches), or such a size as corresponds to the typical dimensions of a human wrist. The housing 122 optionally may be provided with conventional wristwatch features, such as a bezel, face, and mechanical movement or digital clock for telling time.

The smartwatch 100 may also include one or more sensor arrays 107 including a sensor array 107. The sensor array 107 may include one or more sensors configured to collect vital biometric information from the wearer, environmental information, and so on. For example, the sensor array 107 may represent an optical sensor 111. The optical sensor 111 may be utilized as a heart rate sensor that measures the heart rate of the user and the changes or variability of the user's heart rate. One or more portions of the sensor array 107 may be located at an inner surface 103 of the housing 122 (i.e., the surface facing the wearer's body during use). For example, the sensor array 107 may include one or more optical sensors 111 located approximately centrally on the inner surface 103 of the housing 122, and oriented to direct one or more spectra of light from the inner surface 103 towards the wearer's body. The optical sensors 111 (or other sensors of the sensor array 107) may include one or more emitters and receivers. Other components, such as antennas, waveguides, amplifiers, pulse generators, may also be utilized by the sensor array 107. The optical sensors 111 may direct the light, wireless signals, or other emissions at a 90° angle to the inner surface 103, or at an angle less than or greater than 90°. Any desired patterns, numbers, angles, emitters/receivers, settings, and configuration of optical sensors 111 and associated light sources may be used. Light emitting diodes (LEDs) may be utilized as optical transceivers for the optical sensors 111, but other light sources or emitters may be used in other embodiments. For example, one or more laser sensors may be utilized. The optical sensors 111 may also be referred to as optical transceivers, emitters, and receivers.

The sensor array 107 may include any number pressure, motion, optical, mechano-acoustic, phonocardiograph (PCG), and photoplethysmography sensors. In another embodiment, the inner surface 103 may include one or more radar detectors for detecting blood flow and variability to detect the heart rate of the user. For example, the smartwatch 100 may measure the heart rate of the user using ultra-wideband (UWB) impulse radar. UWB impulse radar may utilize low amounts of power and is harmless to the human body as utilized by the smartwatch 100.

The optical sensors 111 may emit light or wireless signals at one or more wavelengths or spectrum. For example, a first group/components set of one or more of the optical sensors 111 may emit light primarily at about 350-450 nanometers (green light), a second group of one of more of the optical sensors 111 may emit light primarily at about 605-750 nanometers (red light), and a third group of the one or more optical sensors 111 may emit light primarily at about 850-1020 nanometers (infrared light). The wavelengths or signals of each group may be clustered together or distributed among the other groups. The different groups of wavelengths and/or signals may be operated simultaneously or separately, as desired. For example, electromagnetic phase-shift spectroscopy may be utilized as is known in the art. Low-energy electromagnetic waves may be broadcast utilizing any number of transmitters/emitters, transmitters, receivers, transceivers, antennas, and so forth. In one example, the red light and infrared light groups may be alternatively activated to operate in a manner to cause oxyhemoglobin and deoxyhemoglobin in the blood to absorb the different light energies for measurements, and these energy levels may be compared to determine blood oxygen saturation, using techniques known in the art.

The optical sensors 111 are positioned and oriented to both emit and receive back signals reflected from the user's body. The optical sensors 111 may measure light reflected from the user's body to detect the presence, amplitude, phase, attenuation/impedance, and other characteristics of reflected light. Optical sensors, photodiodes, or other light receivers which produce a voltage, current, or signal proportional to the amount of impinging light energy may be utilized. The one or more emitters and receivers of the optical sensors 111 may be positioned in any number of patterns.

The receivers of the optical sensors 111 may be tuned to detect and measure particular wavelengths of light. For example, a first group of optical receivers may have a band-pass filter that only transmits/receives light at a range of about 350-450 nanometers (green light), a second group of optical receivers may have a band-pass filter that only transmits/receives light at a range of about 605-750 nanometers (red light), and a third group of optical receivers may have a band-pass filter that only transmits/receives light at a range of about 850-1020 nanometers (infrared light). As another example, one or more of the receivers may include a multi-band “knife-edge” or narrow band filter that allows light at multiple discrete wavelengths to pass through (e.g., a filter that transmits light at one or more wavelengths within the range of 605-750 nanometers and one or more wavelengths within the range of 850-1020 nanometers). As still another example, the received or reflected signals may be unfiltered.

The sensor array 107 may be located at any suitable location on the smartwatch 200 including the housing 122 or the band 118. A location at the geometric middle of the sensor array 109 may provide improved shielding against ambient light, but this is not required as other locations may be utilized. The sensor array 107 also may be located on a protuberance or extensions that extends away from the housing 122 relative to the adjacent portions of the inner surface 103, which may make it more likely that the sensor array 107 will rest firmly against the skin. Such a protuberance or extension may act like a fulcrum that remains in contact with the skin as the housing 122 rocks through a range of motion on the wearer's body. The sensor array 107 may also sit flush with the housing 122 on the inner surface 103. In another embodiment, the sensor array 107 may reside within a receptacle, cavity, or hole within the housing 122 to provide additional protection, enhanced angles for applications of light/signals, or to better eliminate contamination from ambient light.

In one embodiment, the inner surface 103 includes contacts for detecting contact of the smartwatch with the wrist, arm, body, or skin of the user. The contacts may be utilized with other sensors to determine user biometrics, determine the status of the smartwatch 100 (e.g., worn, not worn, charging, etc.) relative to the user, and provide environmental information.

The housing 122 may include any number of sidewalls, interior surfaces, exterior surfaces, and structures. The housing 122 may include a unibody structure or joined materials. The housing 122 may also include trim, inserts, separators, dividers, or other components that structurally and functionally enable the different components. For example, the different components may include contacts, buttons, switches, touch interfaces, and so forth.

The smartwatch 100 may include one or more user interfaces, such as displays, user inputs, audio speakers, microphones, haptic feedback devices (e.g., vibrators or tactile probes), projectors, and so on. The housing 122 (or band) may define any number of holes, ports, or outlets for the microphone 112 and speakers 110 to both receive and communicate sound waves. In one example, the outer surface 113 may have a display 102 configured to provide information to the user/wearer or a person assisting the wearer. An exemplary display 102 may include one or more lights, indicators, or displays, such as light emitting diodes (LED), a two-dimensional LED screen, a two-dimensional liquid crystal display (LCD), a touch screen, and soon. The display 102 may be a touch display for providing information and receiving input from the user. For example, the display 102 may be configured to display specific information, such as time of day, biometrics, location information, health/medication reminders, self-care reminders, appointments, and so forth. The display 102 may work in conjunction with the speaker 110, vibrator, lights, contacts, or other user interface components. The display 102 may also include any number of touch-based, optical, or proximity sensors for detecting information from the user and/or environment.

An exemplary input may include a button, such as a capacitive button, a mechanical button, a momentary switch, or the like. The smartwatch 100 may also include dials, switches scroll wheels, or other mechanical components as well as soft buttons configured to be presented by the display 116 for selection based on the mode, activity, configuration, or user preferences implemented by the smartwatch 100. Multiple displays 116 and multiple buttons may also be used. Functions of the displays 116 and inputs 118 are described in more detail below.

The smartwatch 100 includes charging pins 114. The charging pins 114 may be utilized to charge the smartwatch 100 and may also include or incorporate one or more charging ports, communication ports, or the like. The charging pins 114 may be configured to receive a charger that may be utilized while the smartwatch 100 is being worn by the user or when removed. The charging pins 114 may be recessed within the side of the housing 122 to protect the charging pins 114 while the smartwatch 100 is being worn. For example, the charging pins 114 may include three, four, or more pins for charging the smartwatch 100 and performing communications or updates as needed. The charging pins 114 may also include a removable rubber, silicon, plastic, or polymer cover (not shown) to further protect the charging pins 114 from being bent, or subjected to water, dust, exposure, excessive wear, or so forth. The cover may include a tether or attachment point for the cover. The wearable charger may be included with the smartwatch 100 as an accessory. The charging pins 114 may be associated with magnets that may be utilized to connect a charging adapter that may interface with the charging pins 114 to recharge the battery of the smartwatch 100.

The charging pins 114 may also be utilized to perform data communications, including updates, synchronization, downloads, or other communications between the smartwatch 100 and another device. For example, a charging adapter may include a connector for interface with the charging pins 114 on a first end and a USB connector on another end for connection to a computer, tablet, power adapter/wall outlet, or other power source. The housing 122 also may include one or more charger mounts or interfaces that are configured to mate with a portable charging device, as discussed in more detail below.

In another example, a mini-USB (universal serial bus), micro-USB, standardized port, or custom port may be provided on the housing 122 or other portion of the outer surface 113 to selectively connect to a charging and/or communication cable. As another example, a dedicated charging port (not shown) may be provided on the housing 122, sensor array 107, band 118, or the outer surface 113. In another embodiment, the smartwatch 100 may also utilize inductive chargers that utilized one or more magnets or interfaces integrated with the charging pins 114 to properly align the smartwatch 100 with a charging device, cable, cord, or so forth.

The speaker 110 may communicate the generated sounds through one or more ports or openings in the housing 122. Although not shown, the speaker 110 may include components such as digital-to-analog converters, amplifiers, attenuators, filters, and/or other components necessary for the speaker 110 to convert an electrical signal into a sound wave. In one embodiment, the speaker 110 may include multiple speakers including a tweeter, a mid-range, and a bass speaker or associated component. In another embodiment, the speaker 110 may be configured to generate vibration signals or patterns communicated through the user's ear/head or bone structure. The speaker 110 may communicate measurement information, status of the user, operating mode or status of the smartwatch, performance information (e.g., user, smartwatch, etc.), environmental information, or other applicable information.

The microphone 112 is a component for converting soundwaves into an electrical signal which may be amplified, recorded/saved, or communicated. The microphone 112 may receive voice commands from the user or other authorized parties or receive ambient sounds. The measurements performed by the microphone 112 may be utilized in real-time or saved for subsequent analysis, processing, or communications. For example, sounds may be stored in a memory (not shown) to be processed by one or more processors of the smartwatch 100 to determine the ambient noise, location, individuals/animals near the user, and other applicable information. The microphone 112 may include components, such as analog-to-digital converters, amplifiers, attenuators, filters, and/or other components necessary for the microphone 112 to convert a sound wave into an electrical signal. Voice commands received by the microphone 112 may be used by one or more programs or applications executed by the processor for controlling one or more functions of the smartwatch 100. In one embodiment, sounds, speech, and noises detected by the microphone 112 may be utilized to automatically adjust or tune the mode, settings, configuration, sensors, or other components and functions of the smartwatch 100. For example, the user may be more closely monitored in audio conditions known to cause stress to the user (e.g. large groups, traffic, etc.).

As noted, ambient sounds received by microphone 112 may be used by the processor 14 for calibrating or modifying components and functions of the smartwatch 200. For example, the processor may execute an application stored on the memory 20 adjusting the volume of alerts played by the speaker 110 in loud environments, such as “your heart rate is elevated, please sit down in a quiet area for five minutes.”

It will be appreciated that the various components described as being part of the housing 122 may alternatively be moved to the band 118, or the band 118 and housing 122 may be integrated into a single continuous structure. The components and features of the smartwatch 100 may also be integrated into a band only form factor with integrated smaller displays and so forth.

The smartwatch 100 may include any number of transceivers for communicating with wireless devices 196. The wireless devices 196 may represent any number of smart/cell phones, tablets, computers, beacons, identification tags, network devices, servers, routers, hubs, or so forth. The wireless devices 196 may receive communications from the smartwatch 100 at least in part wirelessly. Any number of wireless signals may be utilized, such as Wi-Fi, Bluetooth, cellular signals, Zigbee, and myriad other signals, protocols, standards, and/or variations. Wireless signals may be combined with any number of hardwired networks, signals, and protocols. The wireless devices 196 may also include servers. The smartwatch 100 may operate with one or more servers to form a system for monitoring the user as herein described.

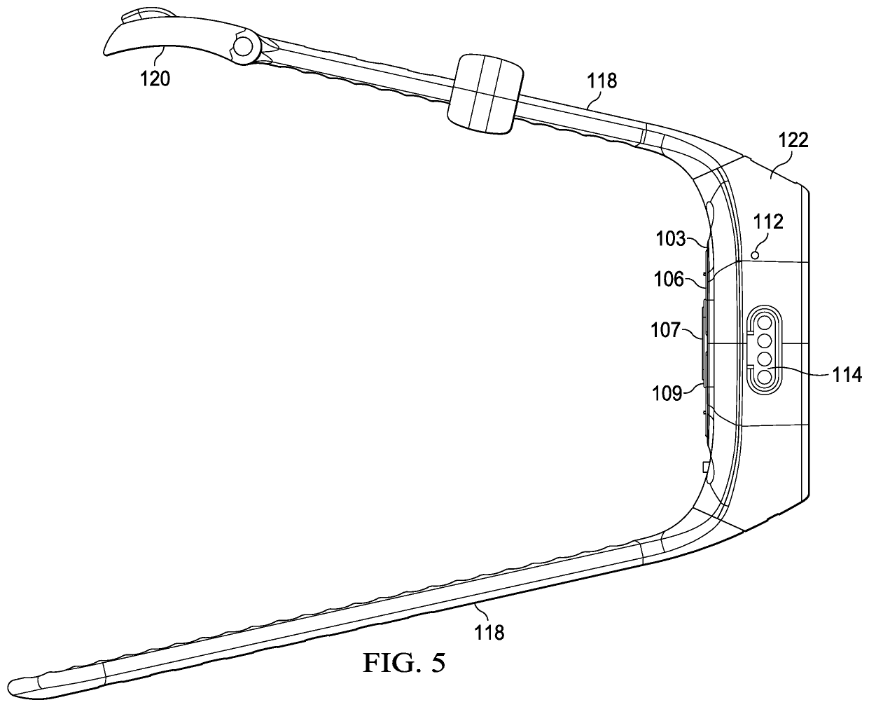

Turning now to FIG. 6 showing different views of a smartwatch 200 in accordance with an illustrative embodiment. The smartwatch 200 may have minor changes in configuration and components as compared to the smartwatch 100 of FIG. 1. The smartwatch 200 may include all or many of the components of the smartwatch 100 of FIG. 1. The smartwatch 200 may represent a single smartwatch 200 or multiple identical smartwatches shown in different positions to further describe the components thereof. The layout and configurations of the smartwatches 100, 200 of FIGS. 1 and 6 may be combined or otherwise utilized. As shown, many of the components of the smartwatches 100, 200 of FIGS. 1 and 6 may be shared. For example, the smartwatch 200 may include four charging pins 114 instead of three charging pins. The optical sensor 104 and the external sensors may be integrated. The microphones 112 may be positioned on the right side of the smartwatch 200 proximate the button 108. The smartwatch 200 may include a pressure sensor proximate the microphones 110 for sensing user applied pressure as well as ambient pressure.

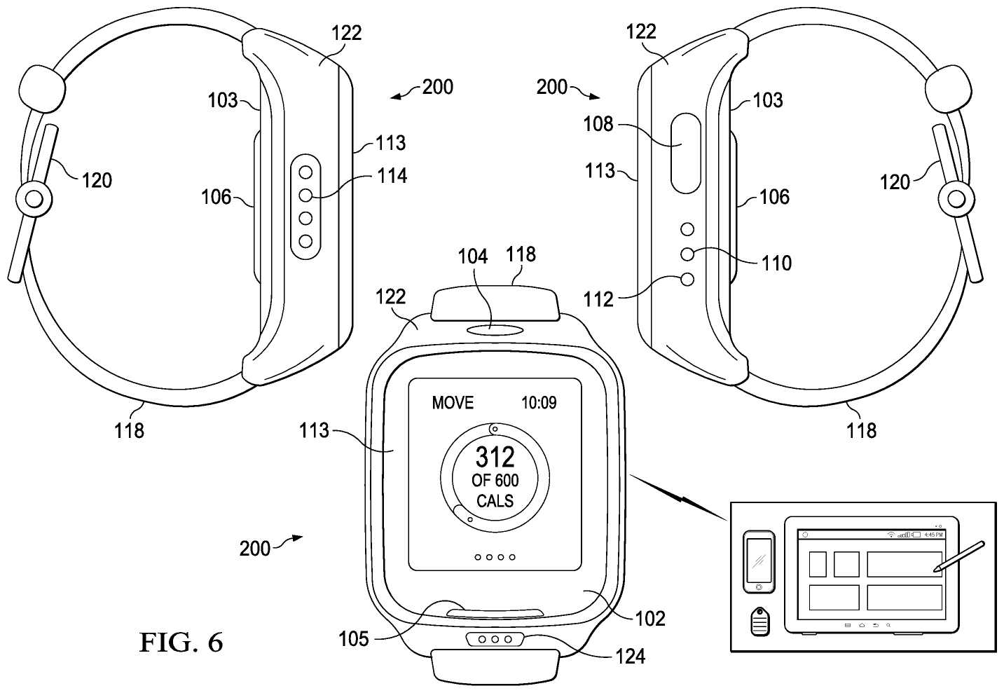

FIGS. 7-10 are a partial view of the smartwatch 200 of FIG. 6 in accordance with an illustrative embodiment. FIGS. 7-10 show a view of the smartwatch 200 with the housing 122 removed or cut-away. The smartwatch 200 may utilize components manufactured by companies, such as Qualcomm, Samsung, Fuji, Taiwan Semiconductor Manufacturing Company (TSMC), and so forth. The components may also be identical or similar for the smartwatch 100 of FIG. 1. The smartwatch 200 may perform traditional fitness tracking processes, such as tracking calories burned (e.g., based on BMI, age, activity), tracking fitness efforts/goals/achievement, encourage sleep/rest, encourage good posture, breathing, and health practices, steps taken, distances travelled, time sitting/standing, and other applicable information utilizing the various sensors (e.g., accelerometers, microphones, gyroscopes, magnetometers, potentiometers, touch sensors/capacitance sensors, global positioning components, transceivers, etc.).

The various components of the smartwatch 200 may be operatively connected to each other and/or one or more processing units (not shown). Any number of traces, busses, wires, fiber optics, cables, wireless interfaces, or other components may be utilized to interconnect the various components of the smartwatch 200. In one embodiment, the display 102 is an AMOLED display with capacitive sensors. The display 102 may also represent liquid crystal displays (LCD), organic light emitting diode (OLED), Micro-LED, or other displays. The display 102 may also be flexible and transparent. Any number of sensors may be integrated into the display 102 itself. The display 102 may be configured to measure information and data (e.g., ambient light, temperature, vibrations, water, humidity, proximity, motion, etc.) regarding the user, environment, or so forth.

The smartwatch 200 may include any number of optical sensors including the optical sensor 104. The optical sensor 104 may be integrated into the housing structure of the smartwatch 200, or band. The optical sensor 104 may perform spectrum analysis of a user's finger or other body part placed on, proximate, or near the optical sensor 104. For example, the optical sensor 104 may determine or confirm pulse oximetry, blood glucose levels, blood oxygenation, pulse information, or other user biometrics. The optical sensor 104 may be a high-resolution camera varying between 5 megapixels and 20 megapixels or more (pixel size may also vary from 2.0, um, 1.5 um, 1.4 um, 1.12 um, to 0.8 um or less). The optical sensor 104 includes multiple components including optics components and image signal processor (ISP) components. For example, the optical components may include a lens, complementary metal-oxide semiconductor (CMOS) or charge-coupled device (CCD) sensor, infrared filter, and motor control sections. Any number of autofocus modules may be utilized including VCM and MEMs.

The ISP may represent a dedicated digital integrated circuit that processes the image data from the CMOS sensors. The optical sensor 104 may connect to a main board, logic, or other components of the smartwatch 200 through a ribbon cable, bus, or other connector.

The electrodes 105, 106 are conductors through which electricity enters or leaves the smartwatch 200. The smartwatch 200 may include a number of electrodes for performing skin conductivity tests, electrocardiograms, alert generation, and so forth. In one embodiment, the electrodes 106 may be positioned on an inner surface 103 and electrode 105 or external surface 109 of the smartwatch 200. For example, electrodes 106 on the inner surface 103 may conduct a current (e.g., test signal/pattern) through a first arm and the user's second arm/hand/finger may be positioned against the electrode 105 on the outer surface 113 of the smartwatch 200. Although a single electrode 105 is shown on the outer surface 113 of the smartwatch 200, the smartwatch 200 (e.g., band 118, latch 120, housing 122, etc.) may include any number of electrodes. Likewise, the electrodes 106 may represent multiple electrodes configured to generate and receive electrical signals (see for Example FIG. 3). The electrodes 105, 106 may then be utilized to measure the electrical activity produced by the heart as it pumps. The electrodes 105, 106 and logic of the smartwatch 200 may check for atrial fibrillation, heartbeat irregularities, and otherwise analyze the electrical activity of the user wearing the smartwatch 200. The other indicators and interfaces of the smartwatch 200 may also include electrode components for performing the measurements herein described (e.g., display 102, button 108, charging pins 114, heart rate sensor 116, latch 120, etc.). The electrodes 105, 106 may also be utilized to measure conductivity, resistance, or capacitance of the user's skin, sweat, blood, tissues, or other portions of the body.

The button 108 may be a switch or selection component utilized to control a process or function of the smartwatch 200. The display 102 may also display any number of virtual controls for controlling the hardware, software, and functionality of the smartwatch 200. In one embodiment, the button 108 may be a power key for turning the smartwatch 200 on/off. For example, selection of the button 108 may be utilized in conjunction with a confirmation selection received by the display 102 (e.g., touch verification, swipe, etc.). The button 108 may also be utilized to change the operating mode of the smartwatch 200. For example, the smartwatch 200 may include a low-power monitoring mode for user status and well-being, fitness tracking for tracking the activity and actions of the user, diagnosis/testing mode for performing one or more tests, a low power location sharing mode for sharing the location of the user, a sleeping mode, a full power mode, an off mode, and any number of other modes that may be needed or required for the user. In one embodiment, the button 108 may also act as an emergency/SOS button. For example, in response to being pressed/activated for longer than 5 seconds, an emergency alert may be communicated. The user may also be prompted to give a verbal request if possible as part of the emergency message.

The smartwatch 200 may also include a number of other buttons integrated with the housing 122 or band 118. Combinations of button selections may be utilized to program the smartwatch 200, provide answer or feedback, send a help request alert message or so forth.

The smartwatch 200 may also include external sensor 124. The external sensor 124 may be a pulse oximeter for measuring the blood oxygenation and saturation of the user (SpO₂). In one embodiment, the user may be prompted to place a finger on the external sensor 124 at predefined intervals, in response to user biometrics or as needed to determine the health and wellbeing of the user. For example, the external sensor may utilize an infrared light, photo detectors to transmit light through a translucent, pulsating arterial bed which is a finger in this case. The external sensor 124 may also be utilized on other portions of the body of the user. The external sensor 124 may utilize transmissive and/or reflective signals to make measurements. The external sensor 124 may also include an ambient light detector. The external sensor 124 may both transmit and receive reflected signals. The housing 122 may include any number of other external ambient light sensors. In one embodiment, the user may send an emergency alert or request for help by simply covering the smartwatch 200 with their hand for a predetermined amount of time. The smartwatch 200 may vibrate and send messages indicating that the emergency message is going to be sent to prevent unwanted messages from being sent.

The charging pins 114 are connectors for charging a battery 130 of the smartwatch 200. The charging pins 114 may be connected to any number of regulators, amplifiers, and other circuits and logic for charging the battery 130. In another embodiment, the charging pins 114 may be replaced by an inductive charger. The inductive charger may allow the batter 130 of the smartwatch 200 to be inductively charged.

The battery 130 may be of any type suitable for powering the smartwatch 200, such as a lithium ion battery. In one embodiment, the smartwatch 200 may be powered by a fuel cell, solar cell, ultra-capacitor, piezo electric generator, thermal generator, or so forth.

Alternative battery-less power sources, such as sensors/receivers configured to receive energy from radio waves or other types of electromagnetic radiation, may be used to power the smartwatch 200 in lieu of an energy source, such as the battery 130. In one embodiment, the processor of the smartwatch 200 may shut down components or features of the smartwatch 200 to preserve the battery life. For example, the smartwatch 200 may shut down the transceivers and non-essential sensors to extend the battery life in a lower power or emergency mode.

The smartwatch 200 may also include light emitting diodes or indicators presented on the display 102 indicating the battery charge, estimated battery life remaining, smartwatch status, user status, alerts, and so forth. For example, a blue light may represent a full battery, a green light may represent a high level of battery life, a yellow light may represent an intermediate level of battery life, a red light may represent a limited amount of battery life, and a blinking red light may represent a critical level of battery life requiring immediate recharging. The user status may also be indicated utilizing the display 102 including LEDs, such as green—good condition, yellow—user may require monitoring, red—the user needs help/assistance/treatment, and blinking red—the user needs urgent and immediate care. In addition, the battery life may be represented by LEDs as a percentage of battery life remaining or may be represented by an energy bar having one or more LEDs. For example, the number of illuminated LEDs represents the amount of battery life remaining in the smartwatch 200. In one example, a connector may interface with the charging pins 114 to recharge the smartwatch 200 through USB charging. In another embodiment, the charging pins 114 may also be utilized for updates to the smartwatch 200.

The vibrator 132 is a small motor that is partially off-balance with respect to a mass distribution attached to the motor's shaft/axis. The irregular weight causes the vibrator 132 to vibrate when activated with a current. For example, the vibrator 132 may represent an eccentric rotating mass vibration motor (ERM), a linear resonant actuator (LRA), or a coin motor. The vibrator 132 may be utilized to provide haptic or tactile feedback or communications to the user.

In one embodiment, the sensor array 107 may represent a heart rate sensor. The heart rate sensor may detect the heart rate of the user, variability, average/median heart rate, and any number of other mathematical or statistical measurements. The heart rate sensor may include the optical sensors, one or more electrical contacts, radar sensors, and other applicable sensors for measuring blood flow/movement, vein/blood expansion and contraction, electrical signals, and other applicable information to measure the heart rate of the user. In one embodiment, a combination of optical and electrical information may be utilized and compared to ensure that the sensor array 107 accurately detects the heart rate of the user. In one embodiment, the optical sensors may utilize photoplethysmogram to measure how much blood the hart is pumping under the surface of the skin. The sensor array 107 may also include pulse oximetry sensors to measure the amount of oxygen in the blood of the user. In one embodiment, the sensor array 107 may work in conjunction with other buttons or components to perform an electrocardiogram (ECG or EKG).

The smartwatch 200 may also include the SIM card slot 109. The SIM card slot 109 may represent a card connector or port for receiving any number of SIM cards (e.g., mini, micro, nano, virtual, etc.) for GSM, PCS, GPRS, SMS, MMS, and other communications protocols, standards, or signals. The SIM card slot 109 may also include a port or be configured to receive one or more SD cards to expand the memory or capabilities of the smartwatch 200. The housing may define an opening or port for adding or removing SIM/SD or other cards to the SIM connector 109. The SIM card slot 109 may also receive a card that provides a transceiver on a chip for proprietary signals or additional channels or capacity for Bluetooth, Wi-Fi, Zigbee, Z-wave, near-field communications (NFC), industrial-scientific-medical (ISM), radio frequency identification (RFID), infrared (IR), NFMI, and so forth. The SIM card slot 109 may also receive additional systems on chip (SoC), additional processing devices, or so forth. In other embodiments, the smartwatch 200 may include a hardware or software SIM that is integrated with the other logic and circuits of the smartwatch 200.

The magnets 136 may ensure a charging adapter is in place (e.g., against, in contact with, or proximate the charging pins 114 when charging the battery 130 of the smartwatch 200. For example, the position and polarity of the magnets may correspond for attachment with a charging/power adapter. In one embodiment, a charger (not shown) may be worn when connected to the charging pins 114. The magnets 136 may alternatively represent metal pieces or a frame so that magnets of the charger may fit against the pins 114.

The smartwatch 200 may also include antennas 138. The antennas 138 may be configured to communicate any number of signals, protocols, or standards. For example, the antennas 138 may be configured for Wi-Fi, Bluetooth, and cellular communications. Other proprietary or other standards may also be implemented by the smartwatch 200. In one embodiment, the antennas 138 may extend into the band 118. As a result, the available space for the antennas 138 may be extended significantly. For example, the antennas 138 may extend into the band 118 on both sides of the frame 122.

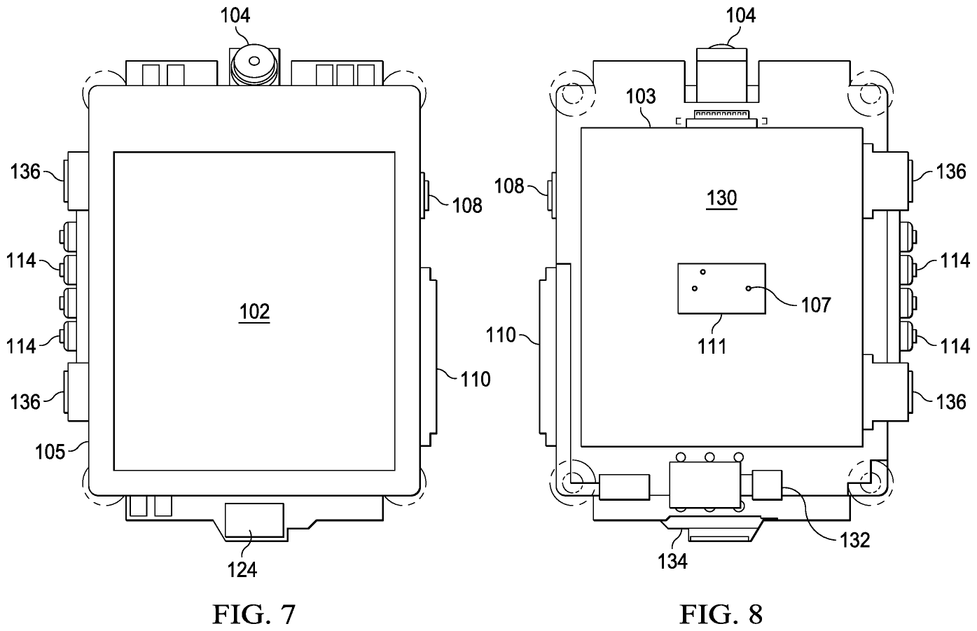

FIG. 11 is a pictorial representation of users wearing a smartwatch 1100 in accordance with an illustrative embodiment. Various users 1102 may utilize the smartwatches 1100 including a toddler 1104, an adult 1106, and an elderly user 1108. The smartwatches 1100 may be utilized by all age groups, genders, and persons without limitation. The smartwatches 100 may be particularly useful for monitoring users 402 that may benefit from or require monitoring.

In one embodiment, the toddler 1104 may represent an infant, toddler, adolescent, or child (0-18+) that require monitoring for health, behavioral, mental, or other issues, problems, disease, tendencies, or so forth. The smartwatches 1100 may be associated with one or more particular locations (e.g., home, school, business, care facility, hospital, etc.). The smartwatches 1100 may also allow for the free travel and movement of the users 1102.

The smartwatches 1100 may represent a single configuration utilized by the various users 1102. In another embodiment, the smartwatches 1100 may represent different configurations utilized for the distinct type of users (e.g., infant/child, young adult, adult, senior, individuals with medical problems, etc.). The smartwatches 1100 may be particularly useful in monitoring a user's status, falls, hydration, and so forth. The smartwatches 1100 may each utilize different types of software (e.g., operating systems, kernels, applications, scripts, instructions, etc.) to monitor and protect the users 1102.

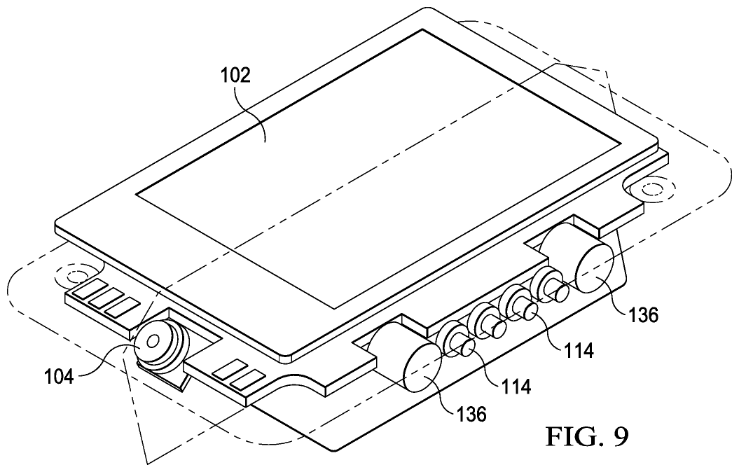

FIG. 12 is a pictorial representation of a block diagram of a smartwatch 1200 in accordance with an illustrative embodiment. The smartwatch 1200 may include any number of operatively connected components including a battery 1208, a logic engine 1210, a memory 1210, a user interface 1214, physical interface 1216, sensors 1218, and transceivers 1220. The smartwatch 1200 may have any number of electrical configurations, shapes, and colors and may include various circuitry, connections, and other components.

All or portions of the components shown and described with regard to the smartwatch 1200 may be included in each of the applicable smartwatches, configurations, or embodiments thereof. For example, some sensors may be included in various smartwatches 1200 for monitoring elderly individuals without inclusion in other embodiments utilized for children. Although not specifically shown, the components of the smartwatch 1200 may be connected or communicate utilizing any number of wires, traces, buses, interfaces, pins, ports, connectors, boards, receptacles, chip sets, transceivers, transmitters, receivers, or so forth. The components may also be integrated in chip sets, boards, programmable devices, or so forth.

The battery 1208 is one or more power storage devices configured to power the smartwatch 1200. For example, the battery 1208 may be a lithium ion battery or other battery type utilized in wearable or small electronics. In other embodiments, the battery 1208 may represent a fuel cell, thermal electric generator, piezo electric charger, solar charger, ultra-capacitor, or other existing or developing power storage technologies. The battery 1208 may also utilize self-powering features, such as self-winding, solar generation, and thermal energy generation (e.g., body heat).

The battery 1208 may be rechargeable or single use. In one example, the battery 1208 may be easily inserted or removed regardless of whether it is rechargeable or not. The physical interface 1216 may include charging pins or a port for charging the battery 1208. The battery 1208 and physical interface 1216 may include circuitry, wiring, hardware, and electronic logic and control systems to control charging of the battery 1208. The charging pins may provide a direct power connection for charging the battery 1208. The charging pins are aligned to allow the charger to be worn with the smartwatch 1200. In an alternative embodiment, the charging pins may also align an inductive charger with an inductive charging receiver of the smartwatch 1200 to charge the battery 1208 inductively.

The logic engine 1208 is the logic that controls the operation and functionality of the smartwatch 1200. The logic engine 1208 may include hardware, software, firmware, circuitry, chips, digital and analog logic, or any combination thereof. The digital logic 1208 may also include programs, scripts, algorithms, processes, and instructions that may be implemented to operate the smartwatch 1200 as well as the components of the smartwatch 1200.

In one embodiment, the logic engine 1208 is one or more processors. The processor may represent any number of microprocessors, digital signal processors (DSP), application-specific integrated circuits (ASIC), field programmable gate arrays (FPGA), or other applicable devices. The logic engine 1208 may utilize information from the user interface 1214, a physical interface 1216, sensors 1218, and transceivers 1220 determine biometric, environmental, and smartwatch 1200 status, information, data, and measurements. Any number of inputs, measurements, data, signals, and external data may be utilized by the logic engine 1208. For example, the logic engine 1208 may make determinations regarding when, where, and how alerts or other communications are sent from the smartwatch 1200. The logic engine 1208 may both send and receive any number of instructions, commands, or data.

In one embodiment, the logic engine 1208 may implement an algorithm allowing the user to associate biometric data as sensed by the sensors 1218 with specific commands, user actions, responses, alerts, and so forth. For example, if the smartwatch 1200 determines the user has fallen based on feedback from the sensors 1218, the user may be asked to verify her physical condition and status to ensure her well-being. A negative response or no response over a designated time period may be utilized to send an emergency communication requesting help, assistance, or a status check. In another example, if the heart rate, blood pressure, or hydration of the user is above or below high and low thresholds, an audible alert may be played to the user and a communication may be sent through the transceivers 1220 to one or more medical professionals, family, friends, or other authorized parties.

The memory 1212 is a hardware element, device, or recording media configured to store data or instructions for subsequent retrieval or access at a later time. The memory 1212 may represent a static or dynamic memory. The memory 1212 may include a hard disk, random access memory, cache, removable media drive, mass storage, or other construct for storing data, instructions, signals, and/or information. In one embodiment, the logic engine 1210 and the memory 1212 may be integrated. The memory 1212 may represent any type of volatile or non-volatile storage components and processes. The memory 1212 may store information related to the status of a user, smartwatch 1200, interconnected electronic devices, smartwatch components, and external devices. In one embodiment, the memory 1212 may execute and display instructions, programs, drivers, operating systems, or code for controlling the components of the smartwatch 1200. The memory 1212 may also store the thresholds, conditions, biometric data, user identification information, authorized parties (e.g., utilization, alerts, etc.), geo-fencing information, passwords, available/accessible/authorized devices, user preferences, health records, biometric statistics, parameters, factors, conditions, and so forth.

The memory 1212 may include modules 1223. The modules 1223 may represent any number of programs, programs, classes, instructions, or sets of instruction that may be implemented by the smartwatch 1200 through execution by the logic engine 1210 or other implementation processes. The modules 1223 may also represent fixed digital logic that may also be integrated with the logic engine 1210 to implement various processes and methods.

In some embodiments, linked or interconnected devices (not shown) may act as a logging tool for receiving information, data, and/or measurements made by the smartwatch 1200. For example, a linked wireless device may download data from the smartwatch 1200 as available, in real-time, or at designated intervals. As a result, the wireless device (i.e. smart phone, tablet, laptop, home computer, designated hub, etc.) may be utilized to store, display, compile, synchronize, and compile data for the smartwatch 1200. For example, the wireless device may display pulse rate, blood oxygenation, blood pressure, blood flow, heart rate variability, temperature, activity, and other applicable biometrics as part of a mobile application executed by the wireless device. The wireless device may also be utilized to receive and display alerts that indicate a specific event or condition has been met. For example, the sensors 1218 may detect that the user is experiencing a cardiac event and may notify an associated wireless device to send the message if available. Alternatively, the transceivers 1220 may send the alerts to any number of designated users/devices. The smartwatch 1200 may offload communications, processing, alerts, and other intensive tasks to an associated communications or computing device to preserve battery power.

The user interface 1214 may include any number and type of components for receiving user input and providing information to the user. In one example, the user interface 1214 may include a display/visual interface, an audio interface, and a tactile interface. The user interface 1214 allows the user to interact with the smartwatch 1200 visually, tactilely, verbally/audibly, biometrically, and through motions. As previously noted, the user interface 1214 may include one or more touch displays for displaying and receiving information and selections from the user. The user interface 1214 may also include any number of physical buttons, switches, dials, scroll wheels, and other components that may be pushed, activated, turned, moved, pulled, touched, or otherwise activate or deactivate components, features, and functions of the smartwatch 1200. Some or all of the buttons of the user interface 1214 may have dedicated functions or controls. For example, one of the buttons may be utilized to turn on and off the smartwatch 1200 as well as activating an emergency request if held for specified time period (e.g., longer than five seconds). A single button may be configured to perform any number of tasks or functions (e.g., switching between modes, powering on/off the smartwatch 1200 or components, entering a different mode, making user selections, sending communications/alerts, activating applications, requesting information, etc.).

As previously noted, the user interface 1214 also includes one or more microphones, speakers, vibrator, and electrical contacts. The microphones may receive audio commands, content, and sounds from the user, measure ambient and environmental noises, and otherwise receive any number of audio and sounds. The speakers may communicate audio content including indicators, alerts, verbal audio, information, and so forth to the user. The speakers may also communicate sound waves that may be “felt” by the user rather than heard. The vibrator may also provide tactile feedback and notices as disclosed with regard to the speakers. The electrical contacts may provide a minor electrical current that may also be utilized to communicate with, alert, or check the status of the client. For example, a small current may be utilized to check hydration, heart rate, skin conductivity, provide alerts, determine if the user is conscious, and so forth.

The physical interface 1216 may represent any number of components and systems for physically interacting with the smartwatch 1200. The buttons of the smartwatch 1200 may be integrated with the user interface and/or physical interface 1216. The physical interface 1216 may also include capacitive or touch sensors. The sensors may be utilized for determining biometrics as well as receiving user input, feedback, and instructions. The physical interface may include the magnets and charging pins for attaching a power adapter for physical or inductive connection. The physical interface 1216 may also include physical interfaces (not shown) for connecting the smartwatch 1200 with other electronic devices, components, or systems, such as a charging system, a smart case, or a wireless device. The physical interface 1216 may include any number of contacts, pins, arms, or connectors for electrically interfacing with the contacts or other interface components of external devices or other charging or synchronization devices. For example, the physical interface 1216 may be a mini or micro USB port. In one embodiment, the physical interface 1216 is a magnetic interface that utilizes the charging pins to couple to an interface of a power system/charger, a wireless device/computing device, or the like. In another embodiment, the physical interface may include a wireless inductor for charging the wireless earpieces 400 without a physical connection to a charging device.

The sensors 1218 may include any number of user or environment sensors. Some of the sensors 1218 may be positioned on the interior (inner/worn side) of the smartwatch 1200 and others may be externally or outwardly facing. The sensors 1218 may include one or more accelerometers, gyroscopes, magnetometers, optical sensors, blood pressure sensors, radar sensors, chemical sensors, pulse oximeters, ECG/EKG sensors, or other physiological, biological, or environment sensors. Further examples of sensors 1218 may include alcohol sensors, glucose sensors, bilirubin sensors, Adenosine Triphosphate (ATP) sensors, lactic acid sensors, hemoglobin sensors, and/or hematocrit sensors. For example, a smartwatch 1200 for an infant may include a bilirubin sensor for monitoring and treating jaundice.

In one embodiment, the sensors 1218 may include radar sensors. As described herein, the radar sensors may be positioned to look toward the user wearing the smartwatch 1200 or externally from the smartwatch 1200. The radar sensors may be configured to perform analysis or may capture information, data, measurements, and readings in the form of reflected signals that may be processed by the logic engine 1210. The radar sensors may include Doppler radar, laser/optical radar, or other radar signals, techniques, and processes.

In one embodiment, the sensors 1218 may include inertial sensors or other sensors that measure acceleration, angular rates of change, velocity, and so forth. For example, inertial sensors may include an accelerometer, a gyro sensor or gyrometer, a magnetometer, a potentiometer, or other type of inertial sensor. The accelerometer may represent single-axis or multi-axis models. The accelerometer may represent microelectromechanical systems (MEMS) and/or sensors. The accelerometer (or alternatively magnetometer or accelerometer) may detect the position and motion of the user and relative portion of the user's body. The inertial sensors may detect deliberate movements for controlling device functions (e.g., shaking, gestures, turning, etc.).

The sensors 1218 include optical sensors. The optical sensors may be utilized to detect user biometrics, ambient light, and so forth. For example, the optical sensor may be configured and utilized as photoplethysmogrpahic (PPG) components that detect blood flow within the user's body. For example, the optical sensor may have an optical emitter that emits light towards the user's skin, and an optical receiver that detects light reflected or absorbed by the blood flowing through the underlying skin and tissue. One or more lasers, LEDs and associated lights sources detectors or components, such as those described above, may be used for this purpose, but other components may also be utilized.

The optical sensors may utilize any number of wavelengths or spectra, such as visible light, infrared (IR), ultraviolet (UV), may be utilized (e.g., X-ray, gamma, millimeter waves, microwaves, radio, etc.). In one embodiment, the spectrometer 442 is adapted to measure environmental wavelengths for analysis and recommendations, and thus, may be located or positioned on or at the external facing side of the wireless earpieces 400.

As the volume of blood in the tissue changes during each heartbeat pulse, the receiver may generate a current or voltage having a waveform corresponding to the change in flow, pressure, volume, or so forth (e.g., PPG data). The heart rate, respiration rate, blood pressure, pulse oximetry, and other biometrics may be measured over time. The sampling rate and size of the data sets may vary based on accuracy, resolution, and power utilization that is required. For example, comprehensive data sets may be gathered based on predetermined events, based on the user status, a schedule (e.g., user based, default, set by a medical professional). The sensors 1218 may utilize multiple measurements to ensure the accuracy of detected biometric data. In one embodiment, the sensors 1218 and logic engine 1210 may utilize algorithms to determine biometric data. For example, sample sets may be analyzed in real-time or subsequently. The logic engine 1210 may utilize mathematical and statistical processing for the biometrics including Fourier transforms, autoregression, demodulation, and so forth. It will be appreciated that any suitable algorithm may be used to estimate pulse rate, respiration rate, oxygen level, and other biometrics, and various such algorithms.

The smartwatch 1200 also may utilize the sensors 1218 to discriminate when the smartwatch 1200 is being worn or not worn. Proximity sensors and temperature sensors may be used for this purpose. For example, when relying on a proximity sensor to determine whether the smartwatch 1200 is being worn, a false positive may arise if the device is removed from the person and placed on a surface in contact with the proximity sensor, and the smartwatch 1200 may continue to operate as if it still being worn.

The sensors 1218 may be tuned, biased, offset, calibrated, or otherwise adjusted in real time or after the fact for optimal performance. For example, the sensors 1218 may be adjusted for conditions or factors, such as ambient noise, white noise, temperature differentials, performance degradation over time, hardware/software errors, and so forth. The sensors 1218 may utilize any number of filters, amplifiers, offsets, or so forth to adjust their respective performance. The sensors 1218 may also utilize any number of sampling rates or frequencies. The sampling frequencies may be adjusted based on the activity, user status, designated parameters (e.g., information from medical professionals), and other applicable information. For example, sampling rates may be adjusted up or down automatically or as selected by the user or another authorized party (e.g., person, device, network system, etc.). The sampling rate may be changed to maximize the performance of the sensors 1218 or to preserve the battery 1208. Any number of mathematical or statistical processes may be utilized to enhance the accuracy and readings made utilizing the sensors 1218.

The sensors 1218 may also include a global position system (GPS) component/unit to determine the location of the smartwatch 1200. The GPS component may operate continuously, based on events, at predetermined intervals/time periods, or based on other information. One or more thermometers or temperature sensors may measure the environmental temperature as well as the temperature of the user. Galvanic, proximity, or touch sensors may also be utilized to detect the presence of the user (inner surface) as well as exterior persons, objects, animals, and so forth.

Although not specifically shown, the smartwatch 1200 may include modular units that may be removed, replaced, exchanged, or otherwise updated. As a result, modular sensor unit may allow the smartwatch 1200 and corresponding sensors 1218 to be adapted for specific users, purposes, functionality, or needs. For example, the modular sensor unit may have contacts or interfaces for being connected or disconnected.

The smartwatch 1200 may also include one or more transceivers 1220. The transceivers 1220 are components including both a transmitter and receiver which may be combined and share common circuitry on a single housing. The transceivers 1220 may communicate utilizing Bluetooth, Wi-Fi, NFMI, ZigBee, Ant+, near field communications, wireless USB, infrared, mobile body area networks, ultra-wideband communications, cellular (e.g., 3G, 4G, 5G, PCS, GSM, etc.), infrared, or other suitable radio frequency standards, networks, protocols, or communications. The transceivers 1220 may also be a hybrid transceiver that supports a number of different communications. The transceivers 1220 may also represent independent transmitters and receivers. For example, the transceivers 1220 may communicate with other electronic devices or other systems utilizing wired interfaces (e.g., wires, traces, etc.), NFC or Bluetooth communications. For example, the transceivers 1220 may allow for induction transmissions with the smartwatch 1220. The transceivers 1220 may represent one, two, three, four or more transceivers that may be separate or share components and circuitry. The transceivers 1220 may be utilized to communicate with any number of communications, computing, or network devices, systems, equipment, or components. The transceivers 1220 may also include one or more antennas for sending and receiving signals. The transceivers 1220 may communicate with any number of networks (e.g., personal area networks, body area networks, etc.) or devices. The smartwatch 1200 may include any number of ports for enhancing the logic engine 1210, memory 1212, sensors 1218, or transceivers 1220. For example, the smartwatch 1200 may receive SIM cards, memory cards, and so forth. The SIM cards may enable any number of communications protocols, standards, or signals. Access to the SIM card may be open or locked for enhanced security. The transceivers 1220 may be utilized to update the software or firmware of the smartwatch 1200, synchronize data, send alerts, messages, or other communications.

In operation, the logic engine 1210 may be configured to convey different information using one or more light emitting diodes or other indicators. The components of the smartwatch 1200 may be located on a printed circuity board(s), chips, circuits, programmable logic, stand-alone components, or a combination of components.

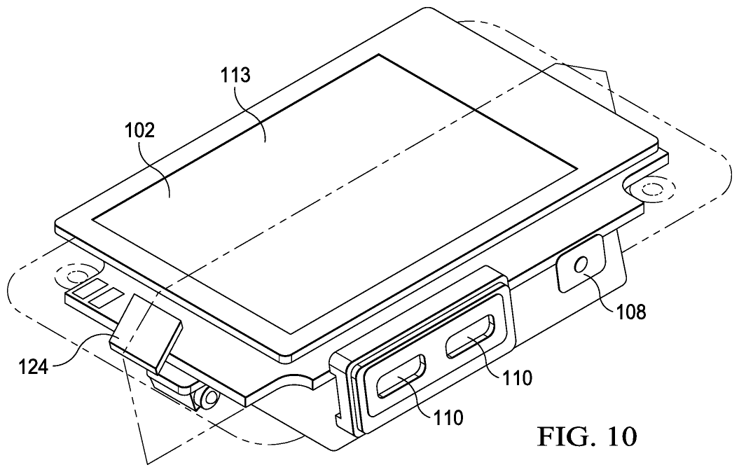

FIG. 13 is a pictorial representation of modules 1223 utilized by the smartwatch 1200 of FIG. 12 in accordance with an illustrative embodiment. In one embodiment, the modules 1223 may be stored in a memory of the smartwatch (i.e., memory 1212 of smartwatch 1200). The modules 1223 may also represent hardware, digital logic, firmware, digital logic, circuits, or a combination thereof. For example, the modules 1223 may be hardwired to perform the functions, processes, steps, features, and actions herein described.

The data of the modules 1223 may be encrypted, coded, or otherwise secured to prevent unauthorized or unwanted access. Any number of passwords, keys, tokens, identifiers, pins, biometrics, or other data/information may be required to access the modules 1223 and their associated data, information, or processes. The user or other authorized agents may specify how, when, and where the information from the modules 1223 may be shared with other users/devices.

The modules 1223 may include a trigger module 122, a health module 1224, a feedback module 1226, an AI/machine learning module 1228, a response module, 1230, and an authentication module 1232. Any number of other modules may also be uploaded, exchanged, downloaded, programmed, utilized, or implemented.

The trigger module 1222 may implement any number of components, actions, features, processes, programs, and other processes (referenced as processes) of the smartwatch 1200. The trigger module 1222 may include a database, library, or references for any number of thresholds, commands (e.g., verbal, tactile, gestural, etc.), measurements, determinations, or so forth that may be utilized to determine a triggered process. The trigger module 1222 may activate, pause, or deactivate any number of processes at any time. As previously noted, the trigger module 1222 may be established based on medical preferences provided by default, a medical provider, an institution/facility, parents/children/guardians, or so forth. In one embodiment, the trigger module 1222 may be activated based on a heart rate that is above or below a designated threshold (145 <HR, 45 > HR). The trigger module 1222 may include any number of other thresholds for biometrics such as impacts, respiration rate, blood pressure, user noises, motion, or so forth.

The health module 1224 manages health information for the user. In one embodiment, user biometrics and health information may be securely stored and accessed utilizing the health module 1224. The health module 1224 may be utilized to determine the status, condition, and well-being of the user. The health module 1224 may indicate the status of a particular condition, disease, malady, abnormality, or other issue. For example, the health module 1224 may determine the user's stability associated with vertigo.

The feedback module 1226 provides input and output for the smartwatch 1200. In one embodiment, the feedback module 1226 may provide information to the user. For example, the feedback module 1226 may provide instructions, commands, or feedback for determining the well-being of the user. For example, the feedback module 1226 may provide instructions for the user to “stand and walk around for five minutes”, “take deep breaths for one minute”, reposition the user's body for better circulation, perform a status test (e.g., balance, vertigo, stamina, endurance, etc.), and perform any number of other processes, steps, or actions. The feedback module 1226 may utilize any of the components or systems of the smartwatch 1200 to provide feedback to the user, associated/linked devices, third-party users or so forth. The speakers, contacts, vibrator, screen/display/lights, or other components of the smartwatch 1200 may be utilized to provide feedback. For example, the feedback module 1226 may provide a verbal indicator that the user “needs to be rolled to prevent bed sores” for a patient that is unable to speak or easily communicate.

The AI/machine learning module 1228 (hereinafter “learning module 1228”) adapts the smartwatch 1200 to the needs and requirements of the user or responsible person(s), institution(s), facilities, agents, medical professional(s), or so forth. In one embodiment, the learning module 1228 may communicate health information to one or more cloud networks or systems. The health information may be analyzed and processed to provide suggestions, actions, activities, and other information applicable to the user. For example, the learning module 1228 may determine baselines, scores, parameters, criteria, and thresholds that may be utilized by the smartwatch 1200. The smartwatch 1200 may adjust the applicable information in real-time, during scheduled updates, based on age, life events, status, condition, diagnosis, and other applicable information.

The response module 1230 controls how and when communications and messages are sent. The communications may be communicated to the user, a medical professional, family/friends/guardians, third parties, monitoring services, institutions, facilities, or so forth.

The authentication module 1212 identifies or authenticates one or more users that may wear the smartwatch 1200. In one embodiment, the smartwatch 1200 may be worn by multiple users. For example, the smartwatch 1200 may be utilized in a care facility to monitor users. As a result, the smartwatch 1200 may be transitioned between different users on any given day (or time period). The authentication module 1212 may utilize biometric information to authenticate the user. The authentication module 1212 may also utilize other applicable information, such as location, activities, actions, behaviors, or other applicable details to identify the user.

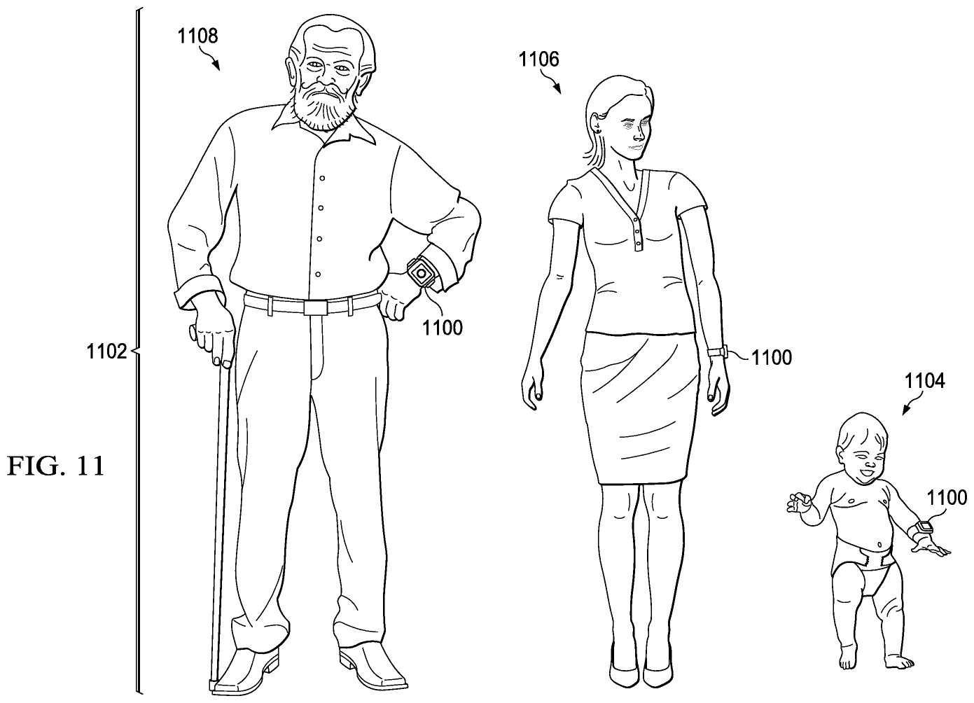

FIG. 14 is another block diagram of a smartwatch 1400 in accordance with an illustrative embodiment. The smartwatch 1400 may represent any of the smartwatches of FIGS. 1-10 and 12. The various embodiments may be combined selectively. The smartwatch 1400 may be is controlled by a computer processor 1402 that is operatively connected to a memory 1404, a power supply 1406, a user interface system 1408, a sensor system 1410 (e.g., sensor array), and a communication system 1412.

The processor 1402 is configured to execute computer-readable instructions stored in the memory 1404. The computer readable instructions may be in a non-transitory format or medium and may be utilized to perform the various processes, functions, and utilization herein described. The memory 1404 may be internal to the processor 1402 or provided as a separate component. The processor 1402 may be a microprocessor having a low power consumption profile. An exemplary processor 1402 is a microprocessor control unit based on the 32-bit ARM Cortex-M4 or A32 core, but any suitable processor may be used. The memory 1404 may be internal to the processor 1402 or external thereto. For example, the memory 1404 may include any suitable digital memory storage system, such as a serial flash memory drive having a 1 Gb capacity. Any number of conforming processors or memories as are known in the art may be utilized.

The power supply 1406 may include a battery, capacitors, a wired power supply leading to an external power source, and so on. The power supply 1406 may be a self-contained battery (e.g., lithium ion or nickel metal hydride) to provide high portability and a long-life cycle. The battery may be rechargeable but may also be single use or replaceable. For example, a quick-change hatch may be utilized to replace the battery. If a rechargeable battery or other rechargeable power supply 1406 is used, the smartwatch 1400 may include a dedicated charge circuit 1414 including wiring, hardware, and electronic logic and control systems to control charging of the power supply 1406, monitor the charge status of the power supply 1406, and so on. The charge circuit 1414 may be configured to interface with an electrical charger that may be utilized even when the smartwatch 1400 is not worn. The charge circuit 1414 may be connected to one or more charging inputs that are configured to receive electric power, such as the charging pins earlier described. One charging input may be a wired charging port on a side of the smartwatch 1400. Another charging input may be an inductive charging receiver, such as a secondary coil connected to a charging circuit that receives electrical energy via inductive coupling, as known in the art.

The user interface system 1408 may include any number and type of devices for receiving user input and providing information to the user. In one example, the user interface system 1408 includes a tactile interface 1414, an audio interface 1416, and a visual interface 1418 (e.g., buttons/touchscreens, one or more microphones and speakers, displays/LEDs).

The tactile interface 1414 may include features that receive and transmit via touch. For example, as noted above, the smartwatch 1400 may include one or more buttons and capacitive/touch displays to receive user inputs. In some embodiments, the electrodes herein described may also be utilized to provide user input and feedback. In one example, a single button is provided on the outer surface to make the device as simple as possible to use in an emergency situation. A single button may be programmed to operate in different modes, depending on the pattern or duration of activation. For example, pressing the button once briefly may turn on the display associated with the visual interface 1418 for a certain period to observe visual information, and pressing it briefly again may turn off the display to conserve power. Pressing the button twice quickly may turn the device on or off. Pressing the button for an extended period, such as three seconds or more, may initiate an emergency alert, as discussed below. Pressing the button for an extended period after initiating an emergency alert may cancel the emergency alert.

In another embodiment, covering the entire smartwatch 1400 for an extended time period may be utilized to send an emergency alert. Multiple different sensors may be utilized to determine the status and intentions of the user. Different inputs for sending alerts may be utilized in the event that the user is incapacitated, heart, or unable to perform standard actions. Alternatively, a single button may have only the single purpose of being pressed to create an emergency alert (and optionally to cancel the emergency alert as well). Where a single button is provided as an emergency alert button, the smartwatch 1400 may be programmed to perform other functions (e.g., setting a clock or customizing the device to the wearer's preferences) via an interface with a smartphone, computer, or other remote terminal.

Other buttons or electrodes may also be provided. Additional buttons may be located and configured to reduce the likelihood that a wearer will confuse those buttons with an emergency alert button. For example, a large emergency alert electrode may be integrated with the crown or exterior surface of the smartwatch 1400, such as show in FIGS. 1 and 6, and additional buttons (e.g., power, mode, programming, etc.) may be provided on the side faces of the outer surface or on the inner surface. The tactile interface 1414 also may include momentum or orientation detecting devices to receive input via physical manipulation of the device. For example, accelerometers, gyroscopes, magnetometers, or other motion sensors may be used to detect deliberate movements for controlling device functions (e.g., hand tapping, gestures, shaking or turning the device over to step backwards in a menu system).

The tactile interface 1414 also may include one or more tactile output devices, such as haptic feedback devices. For example, a motorized actuator with an offset weight may be utilized inside the housing and configured to operate to cause a vibration or movement shift to provide tactile information to the wearer by vibrating the housing. Such feedback may include, for example, vibrating continuously or in a repeating pattern to indicate when an emergency alert has been called, vibrating briefly to indicate when user input has been received, vibrating to indicate certain observed conditions have been met (e.g., pulse rate above a certain level), and so on. The tactile interface 1414 may also utilize small electric currents communicated through one or more electrodes to alert the user, provide feedback, or otherwise communicate with the user.

The audio interface 1416 may include any suitable speaker(s) and/or microphone(s). For example, one or more speakers may be provided in the housing and programmed to emit information in the form of audio output. The one or more speakers may include miniature base, made-range, and tweeter speakers. The audio input may include sounds, tones, or verbal feedback indicating that an emergency alert has been activated or deactivated, that an emergency authority is responding to the alert, and so on. The speakers of the audio interface 1416 may also be utilized to communicate any number of daily operational messages (e.g., greetings, medication reminders, alarms, reminders, appointments, status checks, verifications, etc.). The speaker may also be utilized to transmit audio signals and to provide two-way communication (along with a microphone) with emergency responders. For example, when an emergency alert is generated, the smartwatch 1400 may be connected via wireless communications (e.g., Wi-Fi, Bluetooth, a cellular telephone network, etc.) to an emergency services dispatcher (e.g., a local 911 call center), and telephonic communication may be made through a speaker and microphone within the smartwatch 1400.

The visual interface 1418 includes one or more devices to visually indicate information, such as LED screens, LED lights, visual indicators, or so forth. The visual interface 1418 also may include visual user input systems, such as gesture recognition devices or the like.

The sensor system 1410 may include one or more devices configured to evaluate the environment surrounding the smartwatch 1400. The sensor system 1410 may include an optical sensor 1420 having one or more optical emitters and optical receivers. The optical sensor 1420 also may include an ambient light detection circuit, optical filters, and other features.

The optical sensor 1420 may be configured and programmed as a photoplethysmographic (PPG) device that detects blood vessel performance and volumetric flow of blood within the wearer's body at a location adjacent to the smartwatch 1400. For example, the optical sensor 1420 may have an optical emitter that emits light towards the wearer's skin, and an optical receiver that detects light reflected or absorbed by the blood flowing through the underlying tissue. One or more LEDs and associated detectors, such as those described above, may be used for this purpose, but other devices may be used in other examples.

The volume of blood in the tissue adjacent to the smartwatch 1400 changes during each heartbeat pressure pulse, and the optical receiver generates a current or voltage output having a waveform generally corresponding to the change in flow volume. This may be referred to as PPG data. The PPG data from the optical sensor 1420 may be used to provide heart rate information by evaluating the frequency of flow volume peaks. For example, the heart rate may be estimated by counting the number of flow maxima over time. Other information, such as respiration rate, blood pressure, activity level, and other information may be determined all or in part.

The accuracy of the heart rate estimation may depend on the resolution of the waveform and signal quality, which may be affected by the power output and sampling rate of the system. The sampling rate may be a function of the optical emitter activation cycle, the optical receiver activation cycle, the microprocessor's 1402 activation cycle, and so on. Higher sampling rates provide more detailed PPG data, and increase the ability to pinpoint the exact time of each volume flow peak. However, higher sampling rates also require more energy consumption to activate the optical emitter, poll the optical receiver, and perform the necessary data processing to extract each PPG data point.

It has also been found that the accuracy of the heart rate estimation varies with the length of the sample data set. Estimations based on short sample sets (e.g., five or ten heartbeats) can be significantly less accurate than estimations based on longer sample sets (e.g., fifty or sixty heartbeats). However, extremely long sample sets (e.g., one thousand heartbeats) also provide less accurate instantaneous measurements of heart rate because they can include sample data not reflective of the person's current condition. For example, extremely long data sets will react slowly to rapid changes in heart rate and may not accurately register brief, but significant, changes in heart rate. The selection of the sampling rate and data set length can affect the overall performance of the smartwatch 1400 as a heart rate monitor, however, the balancing of such considerations is within the ordinary skill in the art and can be accomplished successfully without undue experimentation. Various known algorithms may be used to this end.

It is expected that users of the smartwatch 1400 may be more satisfied if the smartwatch 1400 is able to begin providing heart rate measurements shortly after being worn or activated. To this end, a two-stage heart rate measuring algorithm may be used. When the smartwatch 1400 is first activated, the processor 1402 begins operating the optical emitter and optical receiver to collect PPG data. During the initial period of activation, the processor 1402 analyzes the PPG data using a first heart rate algorithm based on a relatively short sample set and begins outputting the results of this algorithm as soon as output information becomes available. This provides a relatively inaccurate heart rate estimation shortly after the smartwatch 1400 begins operating. For example, the first algorithm may use a sample set comprising a 5-10 or 5-30 second rolling window of PPG data. Using this algorithm, the processor 1402 can start providing heart rate estimations shortly after the initial window of data collection is complete. This is expected to provide a heart rate estimation that is accurate within about 10 beats per minute (bpm) of the actual heart rate for the period in question.

After a predetermined time has elapsed, the processor 1402 changes to a second heart rate algorithm based on a relatively long sample set (i.e., longer than the first sample set discussed above) to provide a relatively accurate heart rate estimation. The second algorithm may, for example, use a sample set comprising a 10-60, 20-60 or 30-60 second rolling window of PPG data. The rolling window of data used by the second algorithm may begin at the time the smartwatch 1400 is first activated, in which case the data used to perform the second algorithm may overlap the data used to perform the first algorithm. This minimizes the amount of time before the second algorithm takes over and start providing more accurate heart rate estimations. Using this algorithm, the processor 1402 may be able to start providing heart rate estimates about 35 to 40 seconds after the device is activated. The estimate provided by this second algorithm is expected to be accurate within about 1.0 bpm of the actual heart rate for the period in question. Once results from the second algorithm are available, the processor 1402 may stop performing the first algorithm to conserve energy and processing power. The second algorithm may be used for the remaining duration of the smartwatches 1400 use, until it is removed from the wearer or rendered inactive.

The first and second algorithms may incorporate any algorithm that provides frequency data based on a measured waveform. In one example, the first and second algorithms evaluate frequency domain information from the PPG data using analytical processes such as Fast Fourier Transformations (FFT) to extract frequency domain peaks from the PPG data. Such peaks can then be filtered to identify pulse rate candidates (e.g., frequencies outside a certain range can be removed), and the pulse rate can then be selected as a remaining dominant peak. Other alternatives will be apparent to persons of ordinary skill in the art in view of the present disclosure.

The foregoing dual-stage heart rate algorithm process provides results that are likely to be relatively inaccurate during the initial operation period. However, the ability to provide heart rate estimations shortly after activating the smartwatch 1400 is expected to be beneficial to satisfy the wearer's expected desire to measure his or her heart rate shortly after activating the smartwatch 1400.

Data from the optical sensor 1420 also may be used to determine respiration rate. Arterial blood pressure and peripheral venous pressure change during respiration. This variation manifests itself in PPG data as cyclical changes in flow rate that overlap the flow rate change caused by pulsatile variations. The respiration rate typically is significantly slower than the heart rate, which facilitates extracting the flow rate changes attributable to respiration using techniques such as Fourier transforms, autoregression, demodulation, and the like. Like heart rate estimations, such methods rely generally on evaluating a moving window of data. However, it has been proposed to perform real-time estimation of respiration rate using, for example, adaptive infinite impulse response filters.

In another example, the optical sensor 1420 may be operated to detect blood oxygen level. In this case, the optical sensor 1420 may have a first optical emitter in the red-light range, a second optical emitter in the infrared light range, and a single optical receiver. The red and infrared optical detectors are operated asynchronously to irradiate the underlying body tissue, and the optical receiver may be operated continuously to detect the intensity of red and infrared light reflected by the blood in the wearer's body. The signal from the optical receiver s then demultiplexed according to the operation schedule of the two optical emitters, to determine which portions of the detected light intensity are attributable to reflections of the red light, and which portions of the detected light intensity are attributable to reflections of the infrared light. The ratio of red light reflection intensity to infrared light reflection intensity can then be used to determine the blood oxygen saturation level, because oxyhemoglobin and deoxyhemoglobin absorb different wavelengths of red and infrared light. Such techniques, commonly called pulse oximetry, are known in the art.

It will be appreciated that any suitable algorithm may be used to estimate pulse rate, respiration rate, oxygen level, and other vital signs, and various such algorithms are known in the art. Furthermore, examples of smartwatches may not be capable of or may not be programmed to estimating one or more of the foregoing vital signs.

The smartwatch 1400 also may include features to discriminate when the smartwatch 1400 is not being worn. Proximity sensors and temperature sensors may be used for this purpose, but such devices may be relatively susceptible to experiencing false positive readings. For example, when relying on a proximity sensor to determine whether the smartwatch 1400 is being worn, a false positive may arise if the device is removed from the person and placed on a surface in contact with the proximity sensor, and the smartwatch 1400 may continue to operate as if it still being worn.

Examples also may use the optical sensor 1420 to determine whether the smartwatch 1400 is being worn. For example, PPG data generated by the optical sensor 1420 may be processed using a white noise detector to determine whether the data includes the expected characteristics of pulsatile volume flow variations. A white noise filter may include, for example, an algorithm that averages the amplitude value of the optical sensor 1420 data and identifies whether the data includes a regular periodic signal that passes back and forth through the average value within a particular range of frequency values (e.g., 10-15 times per second). Another white noise filter may include a Fourier transform filter that identifies whether the data from the optical sensor 1420 includes significant peaks in certain ranges of the frequency domain suggestive of a human pulse. Other alternatives will be apparent to persons of ordinary skill in the art in view of the present disclosure.

As noted above, the accuracy of estimations based on PPG data received from the optical sensor 1420 is, in part, a function of the overall minimum sampling rate. It is typical to operate a PPG device at relatively high sampling rates (e.g., 512 Hz) to provide the most accurate PPG data possible. However, it is expected that in the context of a fall detection device such high levels of accuracy may not be necessary. Thus, in some examples, the smartwatch 1400 may be operated at a relatively low sampling rate (e.g., 100 Hz). This is expected to conserve battery power and provide longer service life between battery charging. In such an example, the smartwatch 1400 also may be programmed to automatically switch to a higher sampling rate (e.g., 512 Hz) during specific events, such as when an emergency alert is generated. This can provide more detailed information on an as-needed basis.

The quality or usefulness of PPG data from devices operating at relatively low sampling rates (or even those operating at higher rates) may be improved by performing local up sampling on the data. For example, quadratic interpolation may be performed the PPG data from the optical sensor 1420 to generate a curve to fit each PPG heartbeat pulse profile. Such interpolated curve data may be used to better approximate the locations of maxima, minima, or other values within the curve. In one example, a sampling rate of 100 Hz is combined with ongoing 3-point quadratic interpolation of the incoming PPG data to provide an enhanced PPG data curve without requiring a relatively high sampling rate. Other alternatives will be apparent to persons of ordinary skill in the art in view of the present disclosure.

Estimations of vital signs that are evaluated by the smartwatch 1400 may be indicated to the wearer on the visual interface 1418, such as a display. For example, the display may include a multifunctional LED screen having different modes of operation to display different vital sign data. Mode selection may be performed using any suitable input, such as a dedicated mode button or a multifunction button. One or more of the wearer's heart rate, respiration rate, blood oxygen level, or other vital signs may be indicated on the display at any given time. In one example, heart rate and respiration rate may be numerically indicated on the display. A version of the PPG data also may be displayed on the screen in the form of a pulse curve.

The sensor system 1410 may also include a motion sensor 1422, such as a multi-axis accelerometer, to monitor the physical movement of the smartwatch 1400, and thus the wearer. The motion sensor 222 may include, for example, an intelligent, low-power, 3/6/9-axis accelerometer with 12 bits of resolution. The resolution of the motion sensor 222 (i.e., the range, sensitivity, and sampling rate of acceleration readings) may be selected as desired. The MIS2DH MEMS digital output motion sensor available from STMicroelectronics of Geneva, Switzerland is one example of an accelerometer that may be used in embodiments, but other devices may be used.

Various additional sensors may be provided to the sensor system 210. For example, a Global Positioning System (GPS) unit may be integrated into the smartwatch 1400 to evaluate the location of the smartwatch 1400. Such as GPS device may operate continuously, or may be activated at certain times, such as when an emergency alert is activated. A temperature sensor also may be provided to detect the wearer's temperature or an environmental temperature. As another example, a proximity sensor maybe provided to detect whether an object is adjacent the inner surface of the housing. A galvanic skin response sensor also may be provided, if desired. Other alternatives will be apparent to persons of ordinary skill in the art in view of the present disclosure.

The communication system 1412 may include one or more of a wireless communication interface 1424 and a wired communication interface 1426. The wireless communication interface 1424 may include a transceiver (e.g. an integrated transmitting and receiving device or a paired arrangement of a transmitter and a separate receiver), or it may include only a transmitter. The wireless interface 1424 may be operable to communicate directly with one or more emergency service providers. For example, the wireless interface 1424 may include a digital transceiver operating under the Global System for Mobile Communications (GSM) protocol to communicate directly between the smartwatch 1400 and a digital cellular network. The smartwatch 1400 also may include a Subscriber Identity Module (SIM) card slot to receive user credentials or subscription information. The SIM card slot may be user-accessible, but where the smartwatch 1400 is used in institutional settings (e.g., as a fall monitor in a hospital), the SIM card slot may be sealed to prevent ready access. The wireless interface 1424 also may include any number of other communications devices using various different communication protocols. Examples include, but are not limited to: Bluetooth wireless transceivers, Wi-Fi 802.11 transceivers, Near Field Communication (NFC) transceivers, Zigbee transceivers, and radio frequency (RF) transceivers operating in any suitable frequency range, so on.

The wireless interface 1424 may communicate directly with an existing global communication network. For example, GSM modules can establish communications with existing cellular networks, and Wi-Fi communication modules can establish voice-over internet protocol (VoIP) communications, in respective manners that are known in the art. This may be desirable in applications where the smartwatch 1400 is intended to be worn at a variety of different locations. In other examples, it may be necessary to provide an intermediary communication device to communicate with an existing global communication network. For example, the wearable biosensor smartwatch 1400 may have a Bluetooth or NFC communication module that communicates with a cellular telephone or a local network to gain access to a global network. In other examples, the wireless interface 1424 may be configured to connect only to a particular communication network. For example, the smartwatch 1400 may have a Wi-Fi communication module that is configured to communication with a hospital network in which the smartwatch 1400 is used. As another example, the smartwatch 1400 may have a GSM module that is configured to communicate only with a particular network of call centers or medical response facilities. Combinations of these examples may be used, and other variations will be apparent to persons of ordinary skill in the art in view of this disclosure.

The wired interface 1426 may include one or more connectors to interface the smartwatch 1400 with an external processor or communication device. For example, a mini-USB or other port may be provided for establishing a wired communication link with a local computer. The wired interface 1426 also may be used to establish a wired connection to an external portable communication device, such as a smartphone or the like that is carried on the user's person. When connected in this manner, the external portable communication device may be used to send emergency alerts to emergency service providers, and it may not be necessary for the smartwatch 1400 to have a wireless interface 1424 or a wireless communication device may be temporarily disabled to conserve battery power.

The wireless interface 1424 and the wired interface 1426 may be used for various purposes in addition to sending emergency alerts. For example, one or both of the communication interfaces 1424, 1426 may be used to send configuration settings to the smartwatch 1400, to provide software or firmware updates, to transmit data logs, and so on.

The selection of specific devices, electrical connections, drivers and control algorithms for the user interface system 1408, sensor system 1410 and communication system 1412 will be understood by persons of ordinary skill in the art, and need not be described herein. Examples may include all or only some of the devices described above, and other alternatives and configurations will be apparent to persons of ordinary skill in the art in view of the present disclosure.

The wearable biosensor smartwatch 1400 may be configured as a fall detector and emergency alert device. A significant problem in a fall detection system is the ability to differentiate between events that might require medical assistance and events that do not. A high incidence of false positives can reduce the utility of a device, lead to user dissatisfaction, and generate unnecessary medical service costs.

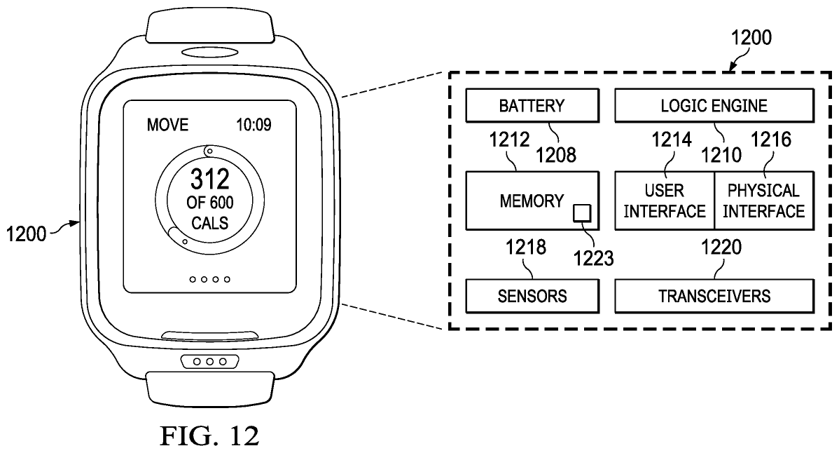

FIG. 15 is a flowchart of a process for generating an automated score decision based on thresholds in accordance with an illustrative embodiment. The process of FIG. 15 may be implemented as a stand-alone process or as part of any number of tests, protocols, processes, devices, systems, equipment, components, assessments, or so forth. In one embodiment, the process of FIG. 15 may be performed by one or more smartwatches or other biosensing wearable devices (e.g., helmets, hearing aids, stickers, bands, sensor packages, hearables, etc.), smart phones, web interfaces, or so forth. For example, the processor(s), microphones, speakers, accelerometers, gyroscopes, timers, and other components of the biosensing as are herein disclosed may be utilized. The process of FIG. 15 may be performed automatically or utilizing user input, interactions, or feedback. The process of FIG. 15 may also be performed by a server or a system, such as a server in communication with the smartwatch through one or more networks.

The process of FIG. 15 may begin by receiving specific inputs for user physiological parameters (step 1502). The user physiology may include height, weight, activity, body dimensions, symmetry, and size, dominant hand, sex, race, medical conditions/issues, and age of a patient. In one embodiment, the smartwatch may prompt the user to provide user input of feedback prior to receiving the data and information of step 1502. The smartwatch may also utilize sensors of the smartwatch to determine information, data, and biometrics, such as activity level, hydration information, heart rate, blood pressure, respiration rate, stress level, user condition/status, location, body position, and other applicable information. The smartwatch may determine the applicable information or receive information and data from external sensors, ambient sensors, additional wearables, external devices, systems, equipment, or components.

In one example, user input of the values for the specified criteria and other criteria may be received through a user interface of the smartwatch. The user interface may be configured to receive audio, visual, tactile, or biometric input. For example, the voice input may be received through one or more microphones and transcribed by the logic of the smart watch to applicable content. The smartwatch may include any number of displays, touch interfaces, buttons, scroll wheels, or other input/output devices for receiving input. The patient or a medical professional may also utilize a web interface available through any number of communications or computing devices, such as web interfaces, applications, cloud systems, cloud networks, or so forth. In one embodiment, the user physiology may represent numbers, menus, drop down menus, or text. For example, the activity may be ranked from low −1, moderate 0, and high 1. The rating scale may also represent a greater range (e.g., 0-10, −5 to 5, A-F, etc.). The wearable device may confirm the values to the user or medical professional audibly, visually, or tactilely or to an associated electronic device. Any number of audible, visual, or other menu options and scales may be presented to the user to receive selections, input, or feedback.

Next, the system calculates a relative index for (step 1504). The relative index may represent any number of indexes, ratios, calculations, or combinations, such as body mass index, height/weight ratios, dominant hand/weight, body dimensions above and below the hip, body density, and so forth. In one embodiment, the BMI may be determined utilizing a formula, such as ((weight (lb.)/height (in))²×703) or weight (kg)/[height (m)]². Any number of English, metric, or other units may be utilized. In addition, various relative indices (e.g., public, proprietary, etc.) may be utilized.

Next, the system assigns values for physiological parameters of the user (step 1506). As noted, the physiological parameters may include any number of factors or parameters, such as BMI, age, race, activity level, hydration, physical condition, medical condition/issues, and so forth. The values assigned may be normalized based on the importance of each of the different factors. Other mathematical processes may be utilized to assign integers, real numbers, or other values. For example, for activity levels the following values or ranges may be utilized low =−1, moderate=0, high=1, for BMI 19-24=1, 25-30=0; and 31-39=−1, for age less than 60=1, 60-70=0, and greater than 70=−1. In one embodiment, any number of additional factors, criteria, conditions, data, or information may be utilized.

Next, the system calculates an impact threshold score (step 1508). In one embodiment, the impact threshold score may be calculated utilizing the values for the physiological parameters including the activity value, age value, body composition, parameters, hydration information, ratios, and diameters, and BMI values previously assigned based on the applicable data and information. For example, the activity value, age value, and BMI value may be added together (e.g., summed values of −5 to 5). The values for the impact threshold score may also be translated or converted to a different scale, data scheme, or so forth. For example, for a score less than −2 the impact threshold score may be 4000, for a score equal to −1 the impact threshold score may be 5000, for a score of 0 the impact threshold score may be 6000, for a score of 1 the impact threshold score may be 7000, and for a score greater than 2 the impact threshold score may be 8000. In addition, the impact threshold score may be increment by 1000 for each integer value above 2. In one embodiment, the impact threshold score may represent register values. The impact threshold scores may correspond to different fall index configurations.

Next, the system establishes thresholds for the smartwatch in response to the impact threshold score (step 1510). The thresholds may be utilized to perform fall prediction analysis. In one embodiment, the register values may be utilized to set thresholds for the user biometrics, sensors, functionality, performance, and processing of the wearable device. For example, timing, current output, sampling rate, user orientation, and sensitivity may be adjusted. The thresholds and/or impact threshold score and associated information may be communicated all or in part to the smartwatch. The smartwatch may utilize logic and or applicable algorithms, programs, and instructions to determine whether the user's biometrics, condition/status, or other data indicates that a fall is imminent or happening.

The smartwatch may communicate an alert through the smartwatch to the user audibly, visually, or tactilely as a warning to the user. The smartwatch may also communicate with any number of authorize users/devices through the transceiver of the smartwatch and available networks. For example, the smartwatch may provide an audio and visual alert for the user to “please sit down and catch your breath”, “take a moment to focus on your breathing”, “please drink some water”, “please call your Doctor”, “call Katie and tell her how you are feeling”, “please take your medicine”, or other applicable messages.

The smartwatch may perform an on-user calibration process for the sensors. The calibration may be performed based on historical information, bias levels, and so forth. The calibration process may also include a reboot or reset. The smartwatch may determine baseline readings for the user to ensure that all measurements are accurate.

The thresholds may be utilized to perform fall risk prediction and detection. In one embodiment, the system may communicate an alert indicating that fall likelihood has surpassed one or more levels, percentages, or so forth. In another embodiment, the system may communicate one or more alerts indicating that a fall has happened to one or more specified users, devices, systems, applications, or so forth.

The illustrative embodiments may also be utilized to measure hydration (or dehydration). Dehydration has a wide range of adverse effects on the human body. Even a small amount of dehydration has been observed to cause any number of issues and physical performance problems affecting short term memory, concentration arithmetic, motor skills, headaches, irritability, and so forth. Long term dehydration may lead to gastrointestinal, kidney, and heart problems, constipation, kidney disease, heart disease, and so forth. For example, the illustrative embodiments may be utilized monitor infants for cystic fibroses, stroke rehabilitation patients, and individuals suffering from a kidney malfunction. The loss of productivity in society has been estimated to be approximately $250 billion. Dehydration may happen due to activity levels and environmental temperatures, but more often users simply do not think about their hydration status to ensure that they are taking in enough fluids.

The smartwatch may utilize any number of sensors, components, or processes for measuring and analyzing hydration in the user. The smartwatch may utilize light-based sensors to detect hydration levels. For example, non-invasive light emissions may pass through the user's skin to measure changes in blood glucose and interstitial fluid that happen with decreasing water volume. For example, the volume of interstitial fluid may decrease below a threshold potentially indicating dehydration. The sensors may also detect changes in the mechanical properties of the user's skin, such as elasticity and texture. The sensors may measure these properties passively (e.g., optically, conductively, etc.) or actively (e.g., actuators, pinchers, motion of the skin against the smartwatch/band, etc.). For example, a miniature set of arms or pinchers may measure elasticity of the skin of the user's wrist or arm.

In another embodiment, the smartwatch may include electrochemical sensors that measure and/or analyze the user's perspiration, blood, or fluids to determination hydration. The sensors may measure fluids as they flow through a portion of the sensor or components of the wearable. For example, mineral content (e.g., sodium, potassium, etc.), conductivity, and pH level of the user's skin/fluids may decrease with dehydration. For example, the pH level of skin may be naturally more acidic when hydrated and slightly more basic when the hydrated. These sensors may be non-invasive or invasive. For example, optical sensors or microneedles may be utilized. Chemical analysis may be utilized to measure mineral content (e.g., sodium, potassium, etc.) and concentration, pH levels, and may be a direct measurement of dehydration. In one embodiment, multiple determinations may be utilized to decrease the risk of false positives based on conditions that may be similar, such as stress, high blood pressure, inherent medical conditions, different body chemistries, and so forth.

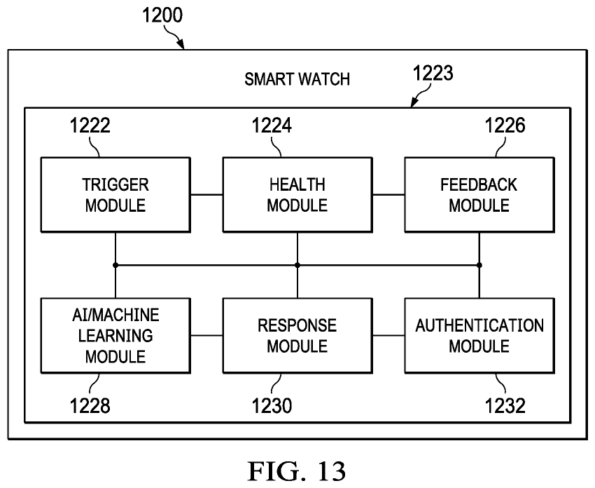

FIG. 16 is a flowchart of a process for creating a hydration profile in accordance with an illustrative embodiment. The process of FIG. 16 may be performed at any time. In one embodiment, the hydration profile may be created during setup of the smartwatch. In another embodiment, the hydration profile may be created as a hydration application is installed, loaded, or executed on the smartwatch. The smartwatch may provide one or more hardware and software interfaces to receive user input and information. For example, a touch screen, buttons, dials, switches, scroll wheels, sensors, or other input devices may be utilized to receive selections. Any number of menus, screens, graphical user interfaces and other applicable information may be utilized. The process of FIG. 16 may be part of a process whereby multiple baseline readings are taken for the user in a baseline, ideal, or default state for the user to ensure accurate comparisons of real-time data.

The process may begin by activating a hydration mode (step 1602). The hydration mode may be activated during step 1602 to perform a configuration or setup process. In other embodiments, the hydration mode may be activated for utilization (not just configuration/setup) in any number of ways including, but not limited to: 1) activation by a user (e.g., user input, selection from a menu, application selection, etc.), 2) occurrence of a specified event (e.g., biometrics, fall event, etc.), 3) at predefined intervals or time periods, 4) in response to a message, signal, command, or external communication, or based on other factors, settings, preferences, locations, conditions, status, parameters, commands, messages, user input, feedback, algorithms, determinations, or so forth.

Next, the smartwatch determines whether the user is hydrated (step 1604). Step 1604 may be performed as a determination or question to the user. In one embodiment, a question is asked during step 1604 to ensure that accurate baseline measurements and readings may be taken. In one embodiment, specific instructions may be given to the user to hydrate himself/herself over a time period (e.g., hours, day, etc.). For example, the user may be required to consume a specified amount of water within a time period. The user may also be encouraged to eat specific foods or drink specified electrolytes to ensure full hydration. The smartwatch may also perform measurements of the user's skin, condition, and status to determine whether the user is hydrated. A combination of determinations and questions may also be utilized. Additional surveys or urine, weight, or other tests may also be performed as part of the determination of step 1604.

If the user is not hydrated, the process ends. In one embodiment, the user may be encouraged to be hydrated before performing the process of FIG. 16. If the user is not hydrated improper data, standards, baselines, readings, and information may be gathered for utilization by the smartwatch, server, cloud system, and other applicable devices. The process may begin again once the user is properly hydrated.

If the user is hydrated, the smartwatch measures conductivity and characteristics of the user's skin (step 1606). During step 1606, the smartwatch measures the conductivity, resistance, reflectivity, characteristics, and property of the user's skin. For example, different users may have different skin pigmentations and conductivity, capacitance, or electrical properties relating to their skin or body. Any number of specialty or generalized sensors may be utilized to perform the measurements of step 1606. Information specific to the user may be utilized to determine hydration, such as age, sex, race, weight, height, body mass index, skin condition (e.g., user specified), and so forth. During step 1606, a hydration value may be assigned to the user.

In one embodiment, a calibration process or steps may be performed during step 1606. For example, the user may be required to perform specific actions or activities. The user may be prompted to place a finger against one or more portions of the screen of the smartwatch to measure skin capacitance, conductivity, reflectivity, and so forth. Hydration profiles may be generated for each user that uses the smartwatch.

Next, the smartwatch creates a hydration profile (step 1608). The hydration profile may include baseline and default readings for the smartwatch. The information in the hydration profile is utilized to determine hydration of the user at any given time that a determination regarding hydration is required. In some embodiments, hydration profiles may be shared for numerous users to determine even more information. The hydration profile may be utilized to more accurately determine the hydration information for the user.

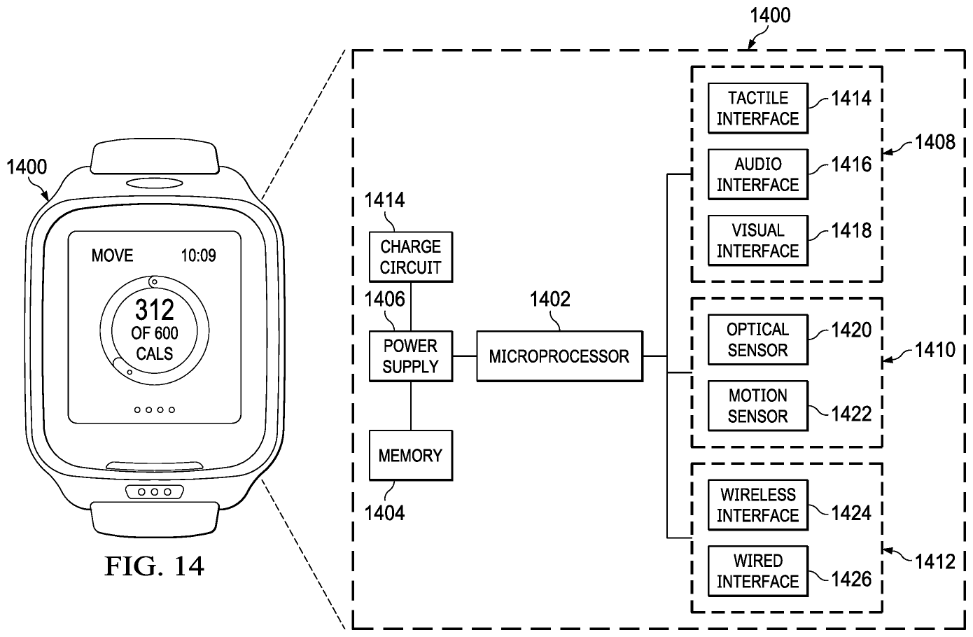

FIG. 17 is a flowchart of a process for performing hydration monitoring in accordance with an illustrative embodiment. The process of FIG. 17 may be performed automatically in response to a time period, user action, event, or so forth. The process of FIG. 17 may also be performed in response to a user request, such as opening an application, selecting a hardware/software button, receiving a verbal, tactile, or gestural command, or so forth. For example, the hydration monitoring may be performed as an overall user monitoring process or fall prevention assessment.

The process of FIG. 17 may begin by requesting a user action (step 1702). The user action may be a request for the user to perform a specific action or activity to initiate hydration testing. In one embodiment, the user action may be pressing a finger against the touch screen/sensor of the smartwatch. The smartwatch may perform measurements utilizing fingers, thumbs, hands, arms, faces, or other portions of the body. The user may also be asked a number of questions, such as “how do you feel”, “do you think you are fully hydrated”, “when did you last drink water”, and so forth. For example, the user may press the worn watch against her forehead, cheek, or opposite arm to initiate the process or monitoring. In another embodiment, the user action may be to ensure that electrodes or contacts on an inner surface of the smartwatch are securely positioned against the skin/body of the user. The user action my require completion of a circuit through small distances (e.g., between electrodes separated by centimeters) or larger distances (e.g., from a first hand of the user through the body of the user to the second hand of the user).

Next, the smartwatch performs conductivity and characteristic testing of the user (step 1704). The measurements may be performed optically and utilizing physical contact with the applicable sensors/displays. The smartwatch may also include a sensor for measuring the content of sweat, moisture, or oils that are part of the user's skin. In one embodiment, the entire screen of the smartwatch may be able to measure information, such as conductivity, resistivity, capacitance, and so forth. The sensors on the underside of the smartwatch may also measure the conductivity and characteristics of the user's skin utilizing light and/or conductors. In another example, a small sensor may require that the finger, skin, or tissue be placed in a specific location, such as a sensor below the touch screen.

Next, the smartwatch compiles measurements from the testing (step 1706). The smartwatch may compile testing measurements, readings, and analysis from one or more sensors or detection devices or components of the smartwatch (e.g., touch sensitive display and wrist-facing sensors). The smartwatch may also compile measurements from other devices in communication with the smartwatch, such as smart clothing, phones, wireless earbuds, glasses, fitness trackers, bands, shoes, and so forth.

Next, the smartwatch analyzes the measurements to generate hydration information (step 1708). The smartwatch may generate any number of hydration ratings, values, information, statistics, data, or information for the user as well as other authorized parties.

The measurements may be analyzed by the smartwatch itself utilizing logic or other information. The measurements may also be offloaded to a connected device or networked system for analysis and generation of the hydration information.

Next, the smartwatch communicates hydration information regarding the user (step 1710). The hydration information may be utilized to alert other user regarding the condition of the user. For example, the communication may be an alert given to the user herself indicating that fluids need to be ingested. The communication may be communicated audibly, textually, visually, or through the other systems of the smartwatch. The communication may also be communicated through another device linked or associated with the smartwatch (e.g., smart phone, television, computer, vehicle, smart wall, electronic glass, etc.). The communication may also be sent to another designated or authorized party or device.

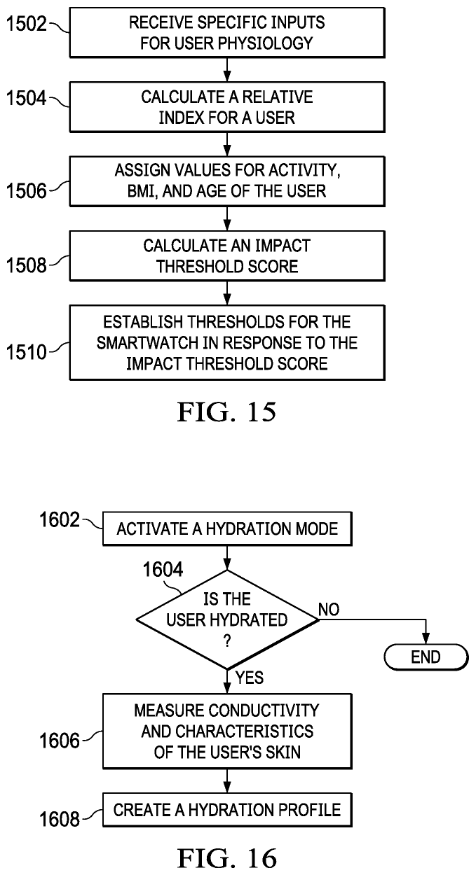

FIG. 18 is a flowchart of a process for automatically performing hydration monitoring in accordance with an illustrative embodiment. The process of FIG. 18 may be performed in conjunction or as an integrated part of the process or steps of FIG. 17. The description for the steps of FIG. 17 and all of the previous Figures is applicable.

The process of FIG. 18 may begin by automatically performing optical testing (step 1802). The testing may be performed periodically, based on an event, based on biometrics, based on a location or previous location (e.g., in the bathroom, kitchen, after returning from the gym/walk, etc.), based on a request or command received from an external source (e.g., a text message sent from an authorized doctor), or so forth. The hydration testing may also be performed at specified time periods or periodic intervals. For example, hydration testing may be performed based on a prescription or request by a doctor or other medical professional associated with the user. In one embodiment, the smartwatch may be prescribed to a user to monitor the user's condition/status, such as hydration, permanently or for a specified time period. The optical testing may be performed from a front (watch face) or inside surface or side of the smartwatch. For example, light emitting diodes (LEDs), sensors, and/or optical receivers may be utilized from either surface. In another example, the smartwatch may be placed in a hydration mode or hydration monitoring mode to automatically perform hydration monitoring and testing for the user.

Next, the smartwatch performs conductivity and characteristic testing of the user (step 1804). During steps 1802 and 1804, any number of tests, steps, algorithms, and processes may be implemented serially, simultaneously, or concurrently. The optical testing and conductivity and characteristic testing of steps 1802 and 1804 may also be performed utilizing the band of the smartwatch. The smartwatch may incorporate a comeometer probe that dielectric value of the skin may be measured from an applied electric scatter field or other applicable fields. Various spectra and wavelengths may be utilized to measure dehydration including water content in skin, blood, and tissues. Any number of physical or wireless connections or signals may be applied to the user by the smartwatch to determine hydration. In one embodiment, multiple different hydration testing techniques may be utilized to ensure accuracy and effectiveness.

Next, the smartwatch compiles measurements from the testing (step 1806). The measurements may measure skin/cell appearance and characteristics, elasticity (turgor), conductivity/capacitance, and so forth. The smartwatch may compile the measurements during a single test or during a series of tests. For example, the smartwatch may test for hydration every five minutes when in a testing mode. The measurements may be compiled in the smartwatch itself or may be sent to an external device associated with the smartwatch, such as a smart phone, tablet, dedicated router, or so forth.

The smartwatch also communicates hydration information regarding the user (step 1808). In one embodiment, the hydration information may be communicated directly to the user/wearer of the smartwatch. For example, a visual and audio message may be communicated to the user using one or more displays and speakers of the smartwatch. The smartwatch may also utilize tactile or electrical communications (e.g., vibrators, electrical currents, etc.). The communications of step 1808 may also be performed through one or more mobile or computing applications, scripts, widgets, or so forth.

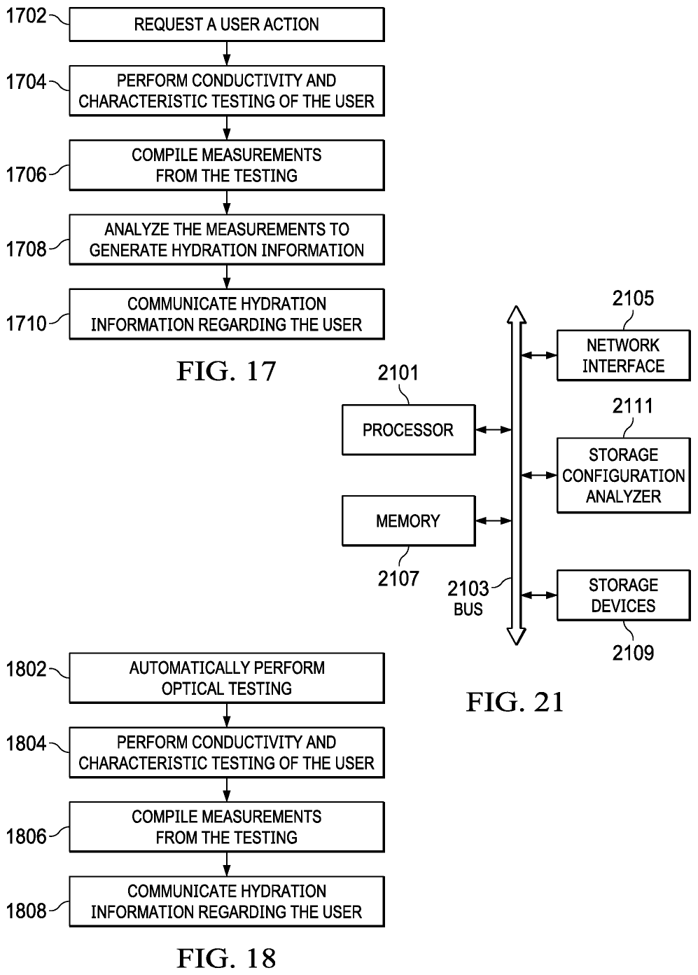

FIG. 19 is a pictorial representation of a smartwatches 1900, 1901 with enhanced bands 1905, 1906 in accordance with an illustrative embodiment. As shown, the smartwatches 1900, 1901 may include enhanced bands 1905, 1906 with latches 1909, 1910 at the ends of the enhanced bands 1905, 1906.

As shown, the enhanced bands 1905, 1906 may include electrical components 1915, such as wires, traces, conductors, pins, capacitors, touch sensors, or so forth. In one embodiment, the electrical components 1915, 1916 may measure biometrics of the user.

The electrical components 1915 may enhance or work in conjunction with the various sensors, components, processors, and other components within bodies 1919, 1920 of the smartwatches is 1900, 1901. The enhanced bands 1905, 1906 may be utilized as electrodes and/or antennas. Utilizing the space available on the bands 1905, 1906 may decrease the space required for electrodes and/or antennas on the body of the smartwatches 1900, 1901. The

In one embodiment, all, or portions of the electrical components 1915, 1916 enhanced bands 1905, 1906, and bodies 1919, 1920 and associated sensors may be disposable or replaced as needed. For example, hydration sensors (or other sensors) may need to be replaced periodically to ensure accuracy. In one embodiment, the electrical components may apply electrical currents or magnetic fields to the body of the user to determine conductivity of perspiration, body (e.g., skin, tissues, blood, etc.) to determine hydration. In one embodiment, the electrical components 1915, 1916 may include optical sensors for measuring and analyzing the optical (e.g., reflectance, diffusion, etc.) characteristics of the user's fluids, body, tissues, or blood. The electrical components 1915, 1916, the enhanced bands 1905, 1906, or the bodies 1919, 1920 may include any number of conductors (e.g., contact pads, conductive straps, etc.), optical or wireless emitters/receivers, flow through/receptacle chemical sensors, and other sensors.

In one embodiment, the electrical components 1915, 1916 may apply currents, voltages, fields or signals to measure conductivity, resistivity, capacitance, or other characteristics of the user's body, fluids, or so forth. As shown the electrical components 1915, 1916 may have different structures or shapes. The different structures and shapes may also correspond to the best transmission and reception characteristics when the electrical components 1915, 1916 represent antennas (e.g., Wi-Fi, Bluetooth, cellular, ZigBee or other signals, standards, or protocols). The electrical components 1915, 1916 may also be utilized to enhance the sensors of the smartwatches 1900, 1901 by detecting user or environmental data or information (e.g., user temperature, humidity, water exposure, proximity to other users/devices/structures, etc.).

The latches 1909, 1910 may be utilized to secure the smartwatch to the user. Any number of mechanical, electromechanical, or other latches, locks, or mechanisms may be utilized to secure the smartwatches 1900, 1901. In one embodiment, the latches 1909, 1910 may be unlocked by a signal or command received from the bodies 1919, 1920 through connectors or wirelessly from any number of devices. Bolts, latches, electromagnets, pins, and/or other mechanisms and components may be integrated as part of the latches 1909, 1910. In one embodiment, the enhanced bands 1905, 1906 may be enhanced with metal, Kevlar, composites, or other materials for preventing the smartwatch from easily being cut or removed. For example, the smartwatches 1900, 1901 may be utilized to track elderly individuals or children that may always or temporarily dislike wearing the smartwatches 1900, 1901 (e.g., discomfort with feeling, dislike of monitoring, etc.). The smartwatches 1900, 1901 may generate an alert or warning if the electrical components 1915, 1916 are cut, damaged, or otherwise interfered with.

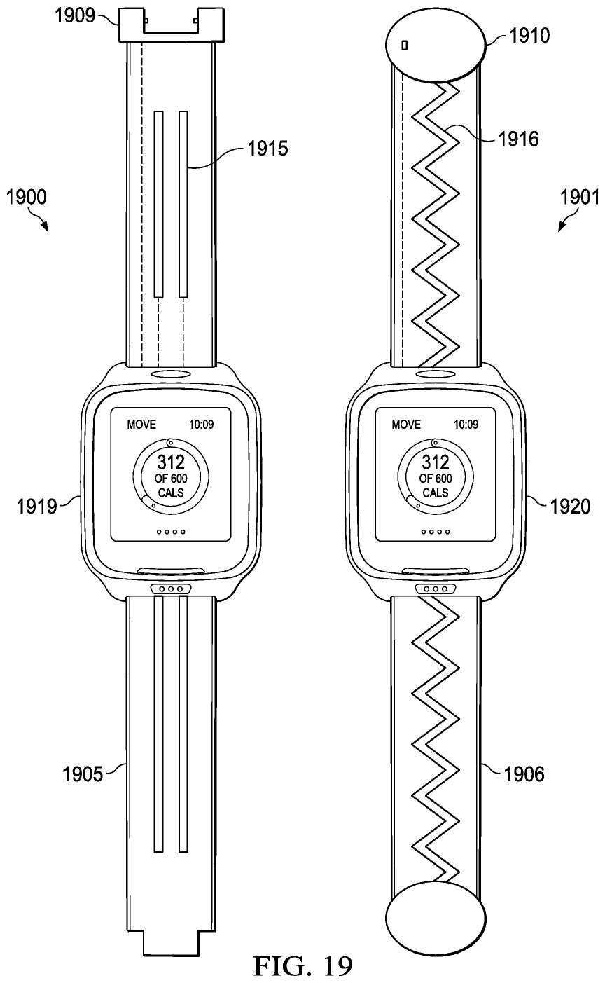

FIG. 20 is a pictorial representation of a smartwatch 2000 measuring hydration in accordance with an illustrative embodiment. The smartwatch 2000 may be representative of any of the smartwatch embodiments as herein described (e.g., FIGS. 1-6, 16). The smartwatch 2000 may utilize a display 2002 to measure hydration based on a touch 2004, press, or fingerprint.

In one embodiment, the display 2002 may measure capacitance, resistance, reflectance, and so forth. The display 2002 may utilize multiple types and forms of measurements for the touch 2004 to measure and characterize the hydration of the user. For example, the display 2002 may be utilized as a comeometer to measure skin hydration. For example, the capacitance of the skin of the user may be determined as a dielectric medium. In another embodiment, a periphery of the display 2002 may include additional capacitance/electrical sensors and/or optical transmitters and receivers for measuring hydration.

The display 2002 may utilize a calibration process to determine standard measurements for individual users. For example, the user may be prompted to provider measurements when fully hydrated (e.g., prompted to drink eight cups of water within two hours). Each different user may have different standard levels of capacitance.

The illustrative embodiments may be utilized to provide instructions for the user to determine when to drink or receive fluids, what to drink, and how much to drink. These instructions may be particularly useful for individuals that are aging, athletes, or individuals that may struggle to track their hydration/dehydration status.

The illustrative embodiments may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, embodiments of the inventive subject matter may take the form of a computer program product embodied in any tangible medium of expression having computer usable program code embodied in the medium. The described embodiments may be provided as a computer program product, or software, that may include a machine-readable medium having stored thereon instructions, which may be used to program a computing system (or other electronic device(s)) to perform a process according to embodiments, whether presently described or not, since every conceivable variation is not enumerated herein. A machine-readable medium includes any mechanism for storing or transmitting information in a form (e.g., software, processing application) readable by a machine (e.g., a computer). The machine-readable medium may include, but is not limited to, magnetic storage medium (e.g., floppy diskette); optical storage medium (e.g., CD-ROM); magneto-optical storage medium; read only memory (ROM); random access memory (RAM); erasable programmable memory (e.g., EPROM and EEPROM); flash memory; or other types of medium suitable for storing electronic instructions. In addition, embodiments may be embodied in an electrical, optical, acoustical, or other form of propagated signal (e.g., carrier waves, infrared signals, digital signals, etc.), or wireline, wireless, or another communications medium.

Computer program code for carrying out operations of the embodiments may be written in any combination of one or more programming languages, including an object-oriented programming language such as Java, Smalltalk, C++ or the like and conventional procedural programming languages, such as the “C” programming language or similar programming languages. The program code may execute entirely on a user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN), a personal area network (PAN), or a wide area network (WAN), or the connection may be made to an external computer (e.g., through the Internet using an Internet Service Provider).

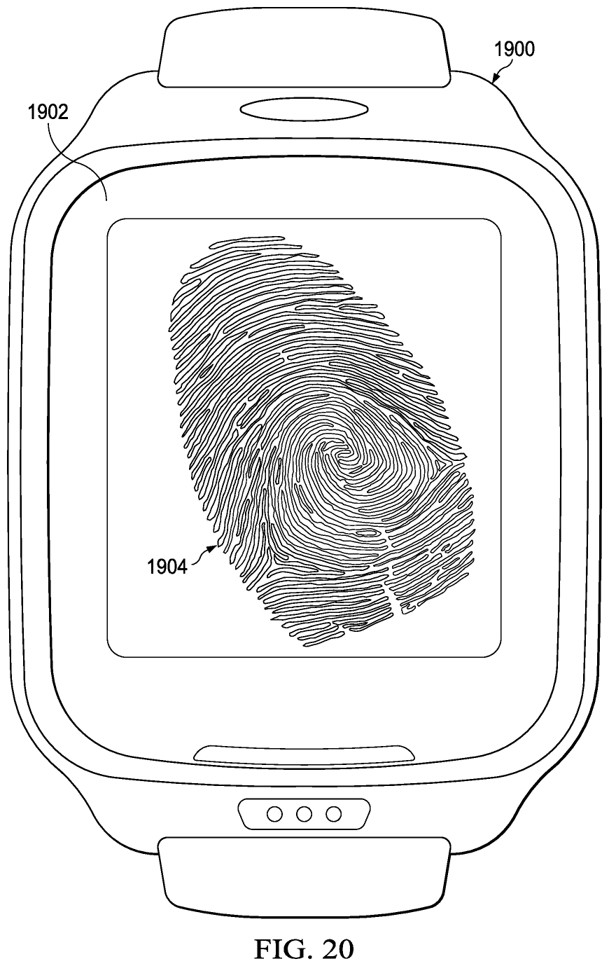

FIG. 21 depicts a computing system 2100 in accordance with an illustrative embodiment. For example, the computing system 2100 may represent a device, such as a server as herein described. The computing system 2100 may utilize any number of encryption and privacy systems to ensure that the data of numerous users is protected and stored securely. For example, the computing system 2100 may comply with HIPAA. Likewise, utilization of the various embodiments of smartwatches may also comply with HIPAA, applicable laws, regulations, industry standards, consumer protections, and so forth. As noted herein, the server may perform any number of processes or steps for receiving, processing, analyzing, and generating inputs, values, analysis, alerts, thresholds, impact threshold scores, and so forth. The computing system 2100 may communicate with a smartwatch to form a monitoring system for one or more users.

The computing system 2100 includes a processor unit 2101 (possibly including multiple processors, multiple cores, multiple nodes, and/or implementing multi-threading, etc.). The computing system includes memory 2107. The memory 2107 may be system memory (e.g., one or more of cache, SRAM, DRAM, zero capacitor RAM, Twin Transistor RAM, eDRAM, EDO RAM, DDR RAM, EEPROM, NRAM, RRAM, SONOS, PRAM, etc.) or any one or more of the above already described possible realizations of machine-readable media. The computing system also includes a bus 2103 (e.g., PCI, ISA, PCI-Express, HyperTransport®, InfiniBand®, NuBus, etc.), a network interface 2105 (e.g., an ATM interface, an Ethernet interface, a Frame Relay interface, SONET interface, wireless interface, etc.), and a storage device(s) 2109 (e.g., optical storage, magnetic storage, etc.). The system memory 2107 embodies functionality to implement embodiments described above. The system memory 2107 may include one or more functionalities that store personal data, parameters, application, user profiles, and so forth. Code may be implemented in any of the other devices of the computing system 2100. Any one of these functionalities may be partially (or entirely) implemented in hardware and/or on the processing unit 2101. For example, the functionality may be implemented with an application specific integrated circuit, in logic implemented in the processing unit 2101, in a co-processor on a peripheral device or card, etc. Further, realizations may include fewer or additional components not illustrated in FIG. 21 (e.g., video cards, audio cards, additional network interfaces, peripheral devices, etc.). The processor unit 2101, the storage device(s) 2109, and the network interface 2105 are coupled to the bus 2103. Although illustrated as being coupled to the bus 2103, the memory 2107 may be coupled to the processor unit 2101.

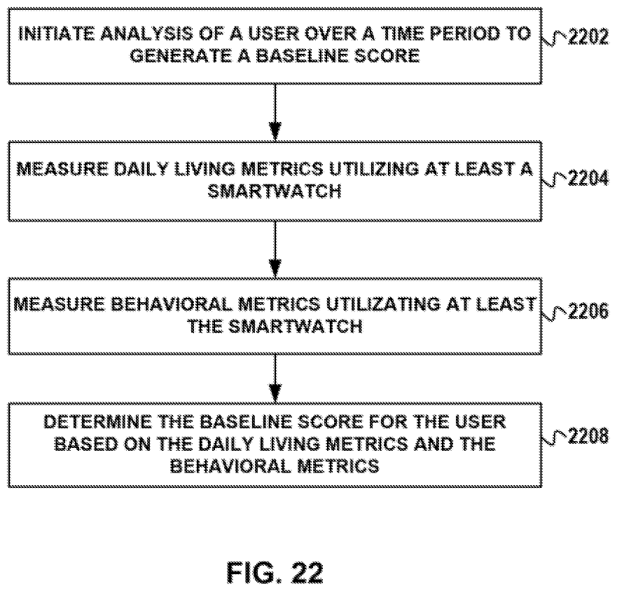

FIG. 22 is a pictorial representation of a flowchart for determining a baseline score for a user in accordance with an illustrative embodiment. The process of FIGS. 22-25 may be performed by a smartwatch alone, the smartwatch in combination with a number of other sensors, and/or the smartwatch in combination with other sensor systems and an intelligent network device (e.g., one or more servers, a server farm, local processing device, laptop, desktop computer, etc.). As noted, the process or methods steps described herein may be performed by a single device or by a number of different associated devices functioning together as a system or platform to perform the method herein described. FIGS. 22-25 may be performed at a single location or across multiple locations. For example, the methods may be performed at a user's residence, family members home, treatment center, monitoring area, hospital, nursing home, or other applicable residential, medical, or commercial setting. The flowcharts and method steps of the described embodiments may be combined in different variations, orders, and formats as is herein contemplated.

The process of FIG. 22 may begin by initiating analysis of a user over a time period to generate a baseline score (step 2202). The user may be wearing a smartwatch as described herein. The analysis may be performed at a location where the user spends most of her time. For example, the process of FIG. 22 may be performed at a home or residence of the user. The time period may represent a week, multiple weeks, month, day, or days. The analysis is performed to determine the physical and mental condition of the user based on actions, movement, activities, independence, self-care, and other applicable factors.

Next, the system measures daily living metrics utilizing at least a smartwatch (step 2204). The daily living metrics may be measured utilizing sensors of the smartwatch. Additional sensor systems may also be utilized (e.g., tags, digital assistants, cameras, smart lights/outlets, etc.). The daily living metrics may include any number of measurements and factors applicable to a user's daily activities and actions. For example, the daily living metrics may measure, report, and capture readings and data associated with a wake time, sleeping, eating, using the restroom, showering/bathing, exercise, and so forth.

Next, the system measures behavioral metrics utilizing at least the smartwatch (step 2206). The behavioral metrics may be utilized to help individuals that may have Alzheimer's, anxiety, dementia, autism, psychological disorders, depression, psychosis, agistation, aggression, disinhibition, sleep disturbances, and other related physical or mental conditions. During step 2206, the user may monitor alcohol, drug, or food consumption, movement, medical interactions, physical pain, and so forth. For example, microphones, speakers, chemical analysis sensors, location sensors, conductivity sensors, accelerometers, gyroscopes, magnetometers, and other sensors of the smartwatch or networked system may be utilized to automatically monitor the behavioral metrics of the user. For example, the behavior of the user may be monitored to ensure the user is standing and exercising enough, taking medications, interacting with others, speaking normally, eating, using the rest room, driving safely, and otherwise performing normal tasks. Recorded patterns of sensor readings may be documented for the user or a number of users to ensure that the proper activities are being performed.

Next, the system determines the baseline score for the user based on the daily living metrics and the behavioral metrics (step 2208). The system measures baseline information because each user is different. As a result, each user will have a distinct baseline score based on their unique status, condition, physiology, medical condition, issues, and so forth. The baselines or may be fixed for a number of users or may be relative or specifically tailored to the user. One or more medical professionals may adjust the baseline score based on additional parameters and information.

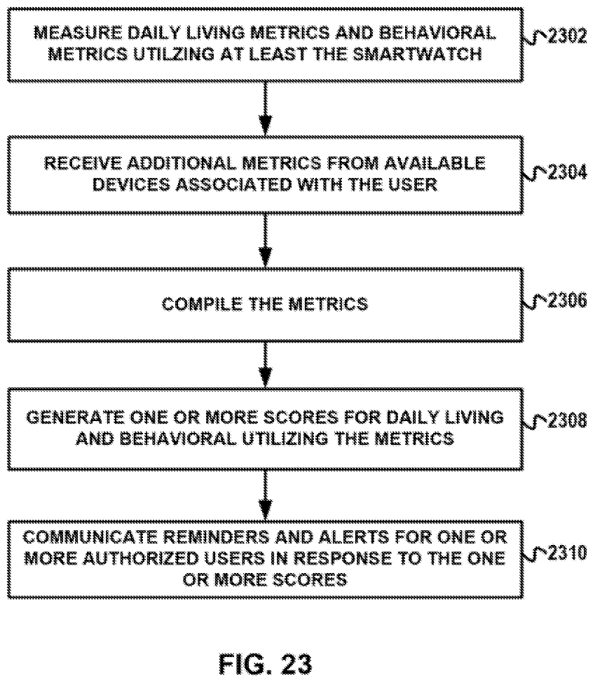

FIG. 23 is a pictorial representation of a flowchart for generating one or more scores for daily living and behavior in accordance with an illustrative embodiment. The process of FIG. 23 may utilize the baseline score is determined in FIG. 22 to better determine one or more scores for a user, such as a daily living score and a behavioral score.

The process may begin by measuring daily living metrics and behavioral metrics utilizing at least the smartwatch (step 2302). The daily living metrics and behavioral metrics may be read by any number of sensors of the smartwatch. Additionally, the system may utilize any number of sensors associated with the location of the user to measure, capture, and measure the various sensor readings, metrics, data, and information (whether in a raw format, semi-processed, or completely processed).

Next, the system receives additional metrics from available devices associated with the user (step 2304). Additional metrics include information received from any number of other sensory components, devices, or systems. The components, devices, or systems AB worn by the user, plaintiff or integrated with the location, temporarily position, or so forth. The system may utilize any number of communications standards, protocols, or formats to capture, send, receive, and compile the daily living metrics, behavioral metrics, and additional metrics all of which may be referred to for simplicity as metrics. For example, communications protocols (and their variations), such as Wi-Fi, Bluetooth, ZigBee, wireless USB, Z-Wave, Long Range Wide Area Network (LoRaWAN), near field communication (NFC), and other similar or developing protocols. The additional metrics may be received by the smartwatch or other portions of the system.

Next, the system compiles the metrics (step 2306). The system utilizes the metrics captured during steps 2302 and 2304 for processing. The metrics may be processed into one or more formats that are usable for determining the condition, status, activity, actions, behavior, and well-being of the user. The metrics may be saved or archived for subsequent reference. For example, the metrics may be sent to a server for long-term storage in association with a user profile of the user.

Next, the system generates one or more scores for daily living and behavioral utilizing the metrics (step 2308). In one embodiment, the scores are based on a numeric scale, such as 0-10, 1-100, word scores, letter scores (e.g., A, B, C, D, F, etc.) or other applicable scores. The scores may be absolute or relative. For example, the user may be compared against other like users utilizing demographics, medical information/status, and other applicable information. In one embodiment, the one or more scores may be based on the baseline score applicable to the user (e.g. the baseline score of FIG. 22). For example, the compiled metrics may be compared against the baseline score of the user to determine trends based on any number of categories, factors, or information.

Next, the system communicates reminders and alerts for one or more authorized users in response to the one or more scores (step 2310). One or more reminders and/or alerts may be sent as part of the method and process of FIG. 23. The reminders and/or alerts may include information indicating how the user is complying or not complying with one or more goals, thresholds, or requirements based on the metrics. The smartwatch may instruct an activity to perform an action or activity as part of the recommendations and/or alerts. For example, the one or more alerts may instruct a user to perform any number of tasks, instructions, activities, or actions to address the potential daily living problems/issues or behavioral problems/issues. Common instructions provided audibly, tactilely, and or visually through the smartwatch and connected devices may include instructing the user to perform breathing exercises, eat some food, take a medication, perform breathing exercises, use the restroom, take a shower, go to sleep, call a friend/family member, play a game, perform a favorite activity or hobby, or so forth.

Alternatively, if the user is doing completely fine, no reminders and/or alerts need be communicated or a status message may be sent periodically. In one embodiment, the reminders and/or alerts may be communicated according to user preferences, settings, or parameters set by the user, caregiver, authorized medical provider, or other authorized user.

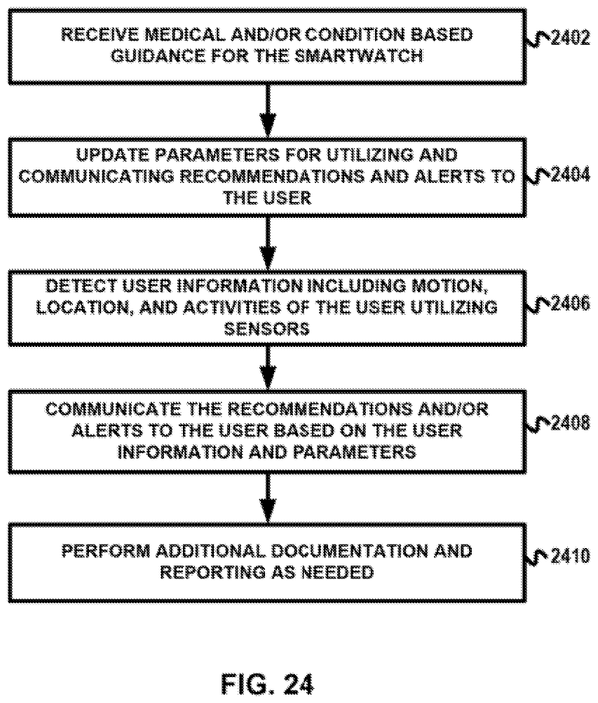

FIG. 24 is a pictorial representation of a flowchart communicating recommendations and alerts in accordance with an illustrative embodiment. The process of FIG. 24 may begin by receiving medical or condition based guidance for the smartwatch (step 2402). The medical an/or condition based guidance may be received from a caregiver, medical professional, government agency (e.g., Centers for Disease Control and Prevention, Food & Drug Administration, etc.), entity (e.g., American Medical Association, AARP, etc.), distribution service, or so forth. In one embodiment, the medical and/or condition based guidance may be received automatically by the smartwatch. In one embodiment, the medical or condition based guidance may be based on ongoing world wide events, medical happenings, and so forth. For example, new medical or condition based guidance may be derived from a pandemic (i.e., COVID-19, flu variations, new strains of viruses, diseases, bacteria, infections, etc.). The medical and/or condition based guidance may include information regarding prophylactic medications, medical treatments, social distancing guidelines (e.g., minimum or maximum distances, congregation sizes, off limits areas/activities, etc.), personal protective equipment (PPE), activity guidelines (e.g., shopping, socializing, travelling, exercising, etc.), and other applicable information.

Next, the system updates parameters for communicating recommendations and alerts to the user (step 2404). In one embodiment, the parameters utilized by the sensors and other systems of the smartwatch may be updated. In addition, the system may update systems utilized for a smart home (also including a nursing home, hospital, care facility, commercial facility, etc.). The parameters may include settings, configurations, thresholds, and other information utilized by the smartwatch and/or system to provide recommendations, warnings, guidance, and feedback to the user. For example, the parameters may provide information regarding social distancing, mask recommendations/requirements, crowd avoidance, location avoidance, activity recommendations/avoidance, action recommendation/activity.

Next, the system detects user information including motion, location, and activities of the user utilizing at least sensors of the smartwatch (step 2406). The smartwatch and/or other sensor systems may be utilized to measure the various motion, location, and activities of the user. For example, a location within a residence may be determined or external to the residence, such as when the user is visiting family or friends, grocery shopping, going to the Doctor, walking for exercise, and so forth. The system may also detect environmental and other user information. In one embodiment, the smartwatch may utilize light sensors to determine whether the user is indoors or outdoors, various transceivers (e.g., Bluetooth, Wi-Fi, NFC, etc.) may be utilized to detect nearby users and their associated smartwatch/devices, a global positioning system of the smartwatch or transceiver may determine the user's exact location (e.g., GPS coordinates, Wi-Fi mapping, wireless triangulation, etc.). The smartwatch may also receive information that is publicly or privately available, such as the attendance or capacity of a nearby grocery store, community event, performance, religious service, gathering, or so forth to determine whether social distancing will be possible.

Next, the system communicates the recommendations and/or alerts to the user based on the user information and parameters (step 2408). In one embodiment, the recommendations and/or alerts may be communicated to influence the user's decisions (e.g., motion, direction, location, activities, choices, alternatives, etc.). In one example, the smartwatch may play an audible and tactile alert, such as “consider wearing a mask at the moment to best protect yourself”, “you should shop at 7:00 am when there are special hours for you”, “please wash your hands”, or “you should consider leaving this area because there are so many people that it makes social distancing nearly impossible.” The system may communicate updated recommendations and/or alerts at any time based on the changing circumstances, environment, motion, location, and activities of the user, other people, and other detectable factors. The system may also communication one or more recommendations and/or alerts through other devices of the system, such as digital assistants (e.g., Siri, Alexa, Google, Cortana, etc.), smart televisions, set top boxes, vehicle systems, speakers, outlets, lights, entertainment/gaming systems, smart home systems, activity tags, and so forth.

Next, the system performs additional documentation and reporting as needed (step 2410). The system may save activities of the user for subsequent reference, archival, or so forth. In one embodiment, the system may save the applicable data and information as determined during FIG. 24 four reference by an authorized user. For example, the applicable data and information may be authorized for use to perform contact tracing. The data and information may also be utilized by a caregiver or other party to ensure that the user's well-being is protected. A smart mask that includes a sensor indicating when it is being worn may communicate with the smartwatch and/or system. For example, alerts that the user is not wearing a mask may be sent to one or more parties as part of the process of step 2410.

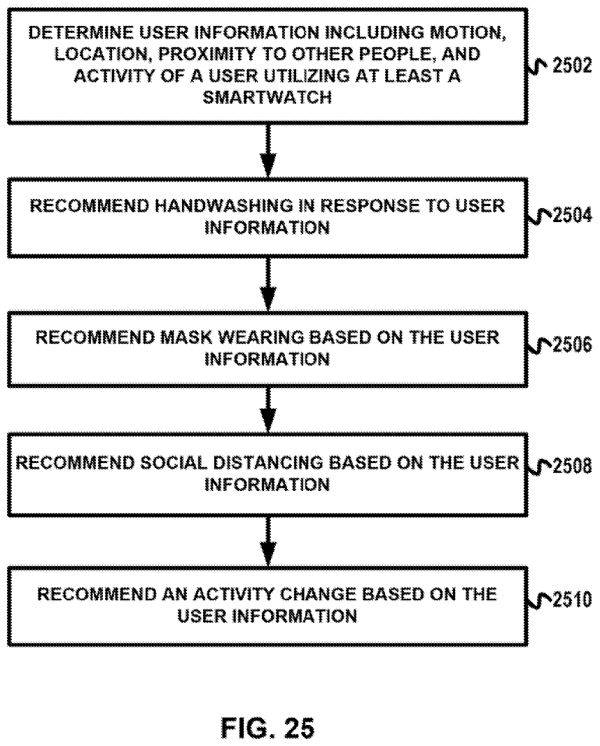

FIG. 25 is a pictorial representation of a flowchart for providing recommendations in accordance with an illustrative embodiment. The recommendations may be performed based on the information performed by any of the previous processes and methods. For example, the process of FIG. 25 may be performed as a portion of FIG. 24 (e.g., step 2406, 2408).

The process of FIG. 25 may begin by determining user information including motion, location, proximity to other people, and activity of a user utilizing at least a smartwatch (step 2502). In one embodiment, the system or smartwatch may process information sensed by the smartwatch and a number of other sensors, devices, components, equipment, or systems. The smartwatch may also request that the user provide information regarding their location, proximity to others, activity, intentions, actions, and so forth.

The smartwatch may also perform processing and analysis of the user information during step 2502 or as a separate step. The user information may be processed to perform any number of recommendations

Next, the system recommends handwashing in response to the user information (step 2504). The handwashing may be recommended to reduce likelihood of the user, caregivers, family, friends, or others from being exposed to unwanted viruses, germs, bacteria, or so forth. For example, handwashing has been shown to slow the spread of viruses, such as Corona Virus. For example, the smartwatch may determine that the user has been outdoors gardening, near other people, or working with animals and may recommend that he wash his hands.

Next, the system recommends mask wearing based on the user information (step 2506). The system may recommend that the user wear a mask in response to proximity to other users (knowing or not knowing the other users are infected with something), environmental conditions (e.g., contaminants in the air), or other factors that may affect the health and well being of the user.

Next, the system recommends social distancing based on the user information (step 2508). The system may recommend social distancing based on the location of the user (e.g., shopping, public performance, meeting, religious gathering, sporting event, concert, etc.). Specific distances (e.g., 6 feet, 4 meters, 10 feet, etc.) may be recommended to the user based on best practice, government guidelines, medical data, or so forth. The system may also recommend social distancing based on individuals or groups known to be sick or at high risk. The user may wear the mask for himself/herself as well as those that the user is around. Other personal protective gear, such as masks, gloves, ear plugs, or gowns may also be recommended by the smartwatch and/or system.

Next, the system recommends an activity change based on the user information (step 2510). A change in activity, location, speed, orientation, proximity to others, or other user condition may be recommended to protect the user. For example, the smartwatch may recommend that the user go to the store at a different time of the day, exercise at a different location, or avoid crowds that have been detected. The steps of FIG. 25 may be recommended and may not all be required or suggested.

The illustrative embodiments may be particularly useful for elderly users, young users, users with disabilities, and others. Real-time data may be captured to provide alerts that may affect daily living and behavior to improve the user's health and well-being.

The features, steps, and components of the illustrative embodiments may be combined in any number of ways and are not limited specifically to those described. In particular, the illustrative embodiments contemplate numerous variations in the smart devices and communications described. The foregoing description has been presented for purposes of illustration and description. It is not intended to be an exhaustive list or limit any of the disclosure to the precise forms disclosed. It is contemplated that other alternatives or exemplary aspects are considered included in the disclosure. The description is merely examples of embodiments, processes, or methods of the invention. It is understood that any other modifications, substitutions, and/or additions may be made, which are within the intended spirit and scope of the disclosure. For the foregoing, it can be seen that the disclosure accomplishes at least all of the intended objectives.

The previous detailed description is of a small number of embodiments for implementing the invention and is not intended to be limiting in scope. The following claims set forth a number of the embodiments of the invention disclosed with greater particularity.

What is claimed is:

1. A method for protecting a user, comprising:
measuring daily living metrics and behavioral metrics utilizing at least a smartwatch worn by a user;
compiling metrics including the daily living metrics and the behavioral metrics;
generating one or more scores for daily living and behavioral utilizing the metrics; and
communicating one or more alerts for one or more authorized users in response to the one or more scores.

2. The method of claim 1, further comprising:
receiving additional metrics from devices associated with the user.

3. The method of claim 1, wherein the daily living metrics and behavioral metrics are read utilizing a plurality of sensors of the smartwatch and one or more questions asked of the user through the smartwatch.

4. The method of claim 1, wherein the one or more alerts provides instructions for the user.

5. The method of claim 1, further comprising:
initiating analysis of a user of a time period to generate a baseline score;
measuring daily living metrics and behavioral metrics utilizing at least the smartwatch; and
determining the baseline score for the user based on the daily living metrics and the behavioral metrics.

6. The method of claim 1, further comprising:
analyzing the metrics against a baseline score for a user to generate the one or more scores.

7. The method of claim 1, further comprising:
receiving medical and condition based guidance for the smartwatch.

8. The method of claim 7, further comprising:
updating parameters in response to the medical and condition based guidance to generate alerts for the user.

9. The method of claim 8, further comprising:
detecting user information including at least motion, location, and activity of the user utilizing the sensors of the smartwatch;

10. The method of claim 9, further comprising:
communicating the one or more alerts in response to the user information and parameters.

11. The method of claim 9, further comprising:
recommending one or more of handwashing, mask wearing, social distancing, and an activity change for the user in response to the user information.

12. A smartwatch for protecting a user, comprising:
a band securing the smartwatch to the arm of the user;
a body of the smartwatch housing logic and at least a portion of one or more sensors, wherein the one or more sensors measure biometrics, motion, location, and activity of the user; and
a user interface in communication with the logic configured to receive input from the user; and
wherein the logic measures daily living metrics and behavioral metrics utilizing at least a smartwatch worn by a user, compiles metrics including the daily living metrics and the behavioral metrics, generates one or more scores for daily living and behavioral utilizing the metrics, and communicates one or more alerts for one or more authorized users through a transceiver in communication with the logic in response to the one or more scores.

13. The smartwatch of claim 12, wherein the logic further initiates analysis of a user of a time period to generate a baseline score, measures daily living metrics and behavioral metrics utilizing at least the smartwatch, and determines the baseline score for the user based on the daily living metrics and the behavioral metrics.

14. The smartwatch of claim 12, wherein the logic generates one or more alerts for communication through the user interface in response to the biometrics exceeding one or more of the thresholds.

15. The smartwatch of claim 12, wherein the transceiver receives additional metrics from devices associated with the smartwatch for compiling with the metrics.

16. The smartwatch of claim 12, wherein the logic analyzes the metrics against a baseline score for a user to generate the one or more scores.

17. The smartwatch of claim 13, wherein the one or more sensors measure user information including at least motion, location, and activity of the user.

18. A system for protecting a user, comprising:
a server configured to communicate through one or more networks, wherein the server receives inputs for user information including at least motion, location, and activity of the user;
a smartwatch measures user information including at least motion, location, and activity for the user utilizing one or more sensors of the smartwatch and communicates the user information from a transceiver of the smartwatch through the one or more networks to the server,
wherein the server compiles metrics including the daily living metrics and the behavioral metrics, generates one or more scores for daily living and behavioral utilizing the metrics, and communicates one or more alerts for one or more authorized users in response to the one or more scores and the user information.

19. The system of claim 18, wherein the server receives additional metrics from a plurality of devices associated with the user including at least the smartwatch, wherein the metrics include the additional metrics.

20. The system of claim 18, wherein the server sends medical and condition based guidance to the smartwatch, wherein the smartwatch updates parameters in response to the medical and condition based guidance received from the server, and wherein the smartwatch communicates the one or more alerts in response to the user information, daily living metrics and the behavioral metrics.
